SEQUENCE LISTING (80 sequences)

SEQ ID NO: 1 - DNA, 20 bp, Artificial Sequence
  Forward primer
agaacatgtgtaagctgcgg

SEQ ID NO: 2 - DNA, 19 bp, Artificial Sequence
  Reverse primer
gttgcctctcactcggttc

SEQ ID NO: 3 - DNA, 20 bp, Artificial Sequence
  Forward primer
gcagaagcgcagatcaaaag

SEQ ID NO: 4 - DNA, 21 bp, Artificial Sequence
  Reverse primer
cggacatgaggctaccatatg

SEQ ID NO: 5 - DNA, 20 bp, Artificial Sequence
  Forward primer
gaaatacctcagcctccagc

SEQ ID NO: 6 - DNA, 20 bp, Artificial Sequence
  Reverse primer
gcgtcacaccattgctattc

SEQ ID NO: 7 - DNA, 21 bp, Artificial Sequence
  Forward primer
atagaaggtgcccaggaaaag

SEQ ID NO: 8 - DNA, 21 bp, Artificial Sequence
  Reverse primer
gtcttcagttcccctccattg

SEQ ID NO: 9 - DNA, 20 bp, Artificial Sequence
  Forward primer
aaacacctcacttccccatc

SEQ ID NO: 10 - DNA, 20 bp, Artificial Sequence
  Reverse primer
accttgcccacatattctcc

SEQ ID NO: 11 - DNA, 21 bp, Artificial Sequence
  Forward primer
ggtacatgagcactgagatcg

SEQ ID NO: 12 - DNA, 19 bp, Artificial Sequence
  Reverse primer
gccacgttgatttccactg

SEQ ID NO: 13 - DNA, 20 bp, Artificial Sequence
  Forward primer
gcacggcttttgttcagatg

SEQ ID NO: 14 - DNA, 19 bp, Artificial Sequence
  Reverse primer
cggttgaaggtgagactgg

SEQ ID NO: 15 - DNA, 18 bp, Artificial Sequence
  Forward primer
atggacgccgaactcatc

SEQ ID NO: 16 - DNA, 19 bp, Artificial Sequence
  Reverse primer
ccaagtccgagaagcagtc

SEQ ID NO: 17 - DNA, 18 bp, Artificial Sequence
  Forward primer
tcatggtgtgggcaaagg

SEQ ID NO: 18 - DNA, 18 bp, Artificial Sequence
  Reverse primer
cgttcagctccttccacg

SEQ ID NO: 19 - DNA, 20 bp, Artificial Sequence
  Forward primer
agaatccagacctgcacaac

SEQ ID NO: 20 - DNA, 19 bp, Artificial Sequence
  Reverse primer
gccggtacttgtagttggg

SEQ ID NO: 21 - DNA, 19 bp, Artificial Sequence
  Forward primer
gagaagccccagaccaaac

SEQ ID NO: 22 - DNA, 20 bp, Artificial Sequence
  Reverse primer
accacacctgacaccttttc

SEQ ID NO: 23 - DNA, 21 bp, Artificial Sequence
  Forward primer
agtagctgtcaaatggccttc

SEQ ID NO: 24 - DNA, 20 bp, Artificial Sequence
  Reverse primer
ttagttcggctttgagggtg

SEQ ID NO: 25 - DNA, 22 bp, Artificial Sequence
  Forward primer
caagaactcgctgatccaaatg

SEQ ID NO: 26 - DNA, 21 bp, Artificial Sequence
  Reverse primer
gctgtacgttccttctccttc

SEQ ID NO: 27 - DNA, 21 bp, Artificial Sequence
  Forward primer
ggcaagacctacatctccaag

SEQ ID NO: 28 - DNA, 19 bp, Artificial Sequence
  Reverse primer
atggcttcctcattgtcgg

SEQ ID NO: 29 - DNA, 18 bp, Artificial Sequence
  Forward primer
catcaaccgcaacgagga

SEQ ID NO: 30 - DNA, 18 bp, Artificial Sequence
  Reverse primer
ggtcatgggtcacgtcag

SEQ ID NO: 31 - DNA, 19 bp, Artificial Sequence
  Forward primer
atggctgacacattcctgg

SEQ ID NO: 32 - DNA, 21 bp, Artificial Sequence
  Reverse primer
catctccttcaacgtctccac

SEQ ID NO: 33 - DNA, 22 bp, Artificial Sequence
  Forward primer
ccctcaaagtaagaccagtagc

SEQ ID NO: 34 - DNA, 22 bp, Artificial Sequence
  Reverse primer
cacagtctccaagaagctctac

SEQ ID NO: 35 - DNA, 22 bp, Artificial Sequence
  Forward primer
ccgctggagacacaatcatatc

SEQ ID NO: 36 - DNA, 20 bp, Artificial Sequence
  Reverse primer
acttcctcaagttgctggtc

SEQ ID NO: 37 - DNA, 20 bp, Artificial Sequence
  Forward primer
cccatgtctccaccttcaag

SEQ ID NO: 38 - DNA, 21 bp, Artificial Sequence
  Reverse primer
ggcttgctcttcatactccag

SEQ ID NO: 39 - DNA, 23 bp, Artificial Sequence
  Forward primer
ggtctatcttaatctggtgctgg

SEQ ID NO: 40 - DNA, 24 bp, Artificial Sequence
  Reverse primer
tggatataggctaaacttcggaac

SEQ ID NO: 41 - DNA, 22 bp, Artificial Sequence
  Forward primer
ctacatgctagacctgtatcgc

SEQ ID NO: 42 - DNA, 21 bp, Artificial Sequence
  Reverse primer
cccactcgtttctggtagttc

SEQ ID NO: 43 - DNA, 20 bp, Artificial Sequence
  Forward primer
gccctcacaaacacctacag

SEQ ID NO: 44 - DNA, 20 bp, Artificial Sequence
  Reverse primer
tcataactccgcccattcac

SEQ ID NO: 45 - DNA, 20 bp, Artificial Sequence
  Forward primer
ggaagcctaactacagcgag

SEQ ID NO: 46 - DNA, 21 bp, Artificial Sequence
  Reverse primer
cagagtcccagatgagcattg

SEQ ID NO: 47 - DNA, 19 bp, Artificial Sequence
  Forward primer
agaaaggctgggcgaagac

SEQ ID NO: 48 - DNA, 20 bp, Artificial Sequence
  Reverse primer
tagcacgtggtcagatgcag

SEQ ID NO: 49 - DNA, 21 bp, Artificial Sequence
  Forward primer
aacctctttgccctgtatgac

SEQ ID NO: 50 - DNA, 20 bp, Artificial Sequence
  Reverse primer
ctgctcattgtcgttggttc

SEQ ID NO: 51 - DNA, 21 bp, Artificial Sequence
  Forward primer
tgcctggacaagatcaatgag

SEQ ID NO: 52 - DNA, 20 bp, Artificial Sequence
  Reverse primer
caggtgtaagtgttgggtcc

SEQ ID NO: 53 - DNA, 21 bp, Artificial Sequence
  Forward primer
ttctgggcacaaacacaaaag

SEQ ID NO: 54 - DNA, 20 bp, Artificial Sequence
  Reverse primer
tcagtcacaatcagggaagc

SEQ ID NO: 55 - DNA, 18 bp, Artificial Sequence
  Forward primer
ctcagctacgccttctcg

SEQ ID NO: 56 - DNA, 23 bp, Artificial Sequence
  Reverse primer
actgtccattttctccttctctg

SEQ ID NO: 57 - DNA, 20 bp, Artificial Sequence
  Forward primer
acaagccatgactcaggatg

SEQ ID NO: 58 - DNA, 20 bp, Artificial Sequence
  Reverse primer
tgtttcactggagcactctg

SEQ ID NO: 59 - DNA, 23 bp, Artificial Sequence
  Forward primer
ccaggttgcaagatttaatgacc

SEQ ID NO: 60 - DNA, 21 bp, Artificial Sequence
  Reverse primer
ttttgatggctctgtggtagg

SEQ ID NO: 61 - DNA, 20 bp, Artificial Sequence
  Forward primer
gcgggctctatcacaaaatg

SEQ ID NO: 62 - DNA, 18 bp, Artificial Sequence
  Reverse primer
tccccattggcattcctc

SEQ ID NO: 63 - DNA, 17 bp, Artificial Sequence
  Forward primer
cacaacgccgagctcag

SEQ ID NO: 64 - DNA, 19 bp, Artificial Sequence
  Reverse primer
ggccggtacttgtagttgg

SEQ ID NO: 65 - DNA, 20 bp, Artificial Sequence
  Forward primer
ctgggtttatgggaaggacg

SEQ ID NO: 66 - DNA, 23 bp, Artificial Sequence
  Reverse primer
cagggagacagaacacataagac

SEQ ID NO: 67 - DNA, 22 bp, Artificial Sequence
  Forward primer
cgagtacatcttcaagccatcc

SEQ ID NO: 68 - DNA, 18 bp, Artificial Sequence
  Reverse primer
tggtgaggtttgatccgc

SEQ ID NO: 69 - DNA, 21 bp, Artificial Sequence
  Forward primer
cctacaccaagagttcgcatc

SEQ ID NO: 70 - DNA, 18 bp, Artificial Sequence
  Reverse primer
tgtgctttcggtagtggc

SEQ ID NO: 71 - DNA, 20 bp, Artificial Sequence
  Forward primer
tggtacccagtgcttttgag

SEQ ID NO: 72 - DNA, 21 bp, Artificial Sequence
  Reverse primer
ctccgatagtccatagcaagg

SEQ ID NO: 73 - DNA, 21 bp, Artificial Sequence
  Forward primer
ctgttcctcaccatccctttc

SEQ ID NO: 74 - DNA, 22 bp, Artificial Sequence
  Reverse primer
caaggagatgagaaacgaggtg

SEQ ID NO: 75 - DNA, 20 bp, Artificial Sequence
  Forward primer
ggtgtcagccttctaatggg

SEQ ID NO: 76 - DNA, 18 bp, Artificial Sequence
  Reverse primer
tcaggcattttcccgtcc

SEQ ID NO: 77 - DNA, 20 bp, Artificial Sequence
  Forward primer
tcagcaactcattcccacag

SEQ ID NO: 78 - DNA, 20 bp, Artificial Sequence
  Reverse primer
gcagggctggtttaggatag

SEQ ID NO: 79 - DNA, 21 bp, Artificial Sequence
  Forward primer
ggactatgagggcaagaactg

SEQ ID NO: 80 - DNA, 21 bp, Artificial Sequence
  Reverse primer
aaatataccgcaccccttcag

(19) United States
(12) Patent Application Publication
(10) Pub. No.: US 2020/0140827 A1
(43) Pub. Date: May 7, 2020

(54) A SUBSTRATE AND METHOD FOR THE GENERATION OF INDUCED PLURIPOTENT STEM CELLS

(71) Applicant: THE QUEEN'S UNIVERSITY OF BELFAST, Belfast (GB)

(72) Inventors: Andriana MARGARITI, Belfast (GB); Marta VILA-GONZALEZ, Belfast (GB); Sophia KELAINI, Belfast (GB); Alan STITT, Belfast (GB)

(73) Assignee: THE QUEEN'S UNIVERSITY OF BELFAST, Belfast (GB)

(21) Appl. No.: 16/617,417

(22) PCT Filed: May 30, 2018
(86) PCT No.: PCT/EP2018/064296

§ 371 Date: Nov. 26, 2019

(30) Foreign Application Priority Data
May 30, 2017 (GB) 1708554.9

(51) Int. Cl. C12N 5/074

(52) U.S. Cl. CPC C12N 5/0696; C12N 2533/54; C12N 2500/38; C12N 2500/44; C12N 2500/90; C12N 2501/105; C12N 2501/115; C12N 2501/125; C12N 2501/14; C12N 2501/15; C12N 2501/155; C12N 2501/16; C12N 2501/165; C12N 2501/2303; C12N 2501/39; C12N 2501/602; C12N 2501/603; C12N 2501/604; C12N 2501/606; C12N 2501/608; C12N 2501/727; C12N 2502/13; C12N 2506/11; C12N 2510/04; C12N 2533/52; C12N 2500/24

(57) ABSTRACT

This disclosure relates a composition and method for promoting the reprogramming of somatic cells to induced pluripotent stem cells, the composition comprising gelatin and laminin. The disclosure further relates to a method of preparing somatic cells for producing induced pluripotent stem cells and a method for producing induced pluripotent stem cells, and thus provides method useful for the production of expanded somatic cells and induced pluripotent stem cells for use in research and therapy. Thus, the disclosure provides a method of preparing somatic cells for producing induced pluripotent stem cells, the method comprising: (i) isolating somatic cells from a sample, and (ii) expanding the somatic cells for a predetermined period of time, wherein the expanded somatic cells express TERT1, as well as a method for producing induced pluripotent stem cells from said expanded somatic cells by (a) introducing genetic elements, optionally episomal genetic elements, that express induced pluripotent stem cells reprogramming factors into said expanded somatic cells and (b) culturing said expanded somatic cells comprising the genetic elements, thereby producing induced pluripotent stem cells.

### TECHNICAL FIELD

This invention relates to a substrate and method for preparing somatic cells for producing induced pluripotent stem cells and a method for producing induced pluripotent stem cells.

### BACKGROUND ART

It has been about a decade since the first discovery of induced pluripotent stem cells (iPS cells). Research into this area has intensified due to the potential usage of iPS cells as a form of personalised treatment for many diseases with a huge emphasis being placed on personalised medicine, in current and future healthcare. A study conducted by Worringer et al. [1] has suggested that efficiency of iPSC formation is as low as 1% when Oct3/4, Sox2, c-Myc and Klf4 reprogramming factors are used. Various studies have attempted to understand why the efficiency of reprogramming between somatic cells and iPS cells is so low in order that improvements to the technique might be made. Numerous studies have looked to ascertain why the efficiency is so low, and have looked to increase the efficiency by trying to enhance reprogramming efficiency. The importance of epigenetic marks and modifications has been examined.

iPS cells have numerous potential applications within healthcare and research. iPS cells can be used for regenerative medicine or cell-based therapies, for modelling mechanisms of disease, for drug screening and cellular toxicity tests.

Transplant of exogenous tissue risks rejection and requires the long-term use of immunosuppressant drugs to ensure “acceptance”. Regenerative medicine would circumvent this problem by using a patient's own cells to create iPS cells and therefore new tissues for transplant.

The potential of iPS cells within personalised medicine is very exciting. In the ten years since Yamanaka and Takahashi first developed iPSCs [2], more is understood about the mechanisms and potential uses. Trials in animal models, including non-human primates, are promising for many diseases and clinical trials are being suggested with an ongoing trial for a patient's macular degeneration. Clinical-grade iPS cells for application in regenerative medicine are being developed. This is a huge step in the move towards human trials and it will be very exciting to see how the use of iPS cells in human personalised and regenerative medicine progresses over the next few years. However, the iPS cells generation is still a long term process with extremely low efficiency which significantly holds back the potential of these cells to be used widely in the clinic. In this study, we have developed a novel, fast and highly efficient approach for generating iPS cells from only a few drops of blood from healthy volunteers and diabetic patients.

This approach is particularly useful in relation to prevention and treatment of vascular disease. The mortality rate for vascular diseases, such as diabetes, is one of the highest around the world, and maintaining the healthy and normal function of the endothelium is of utmost importance for preventing the development and progression of vascular disease. As a result, the main focus and aim of vascular regenerative medicine is the repair and regeneration of damaged cells including the generation of functional endothelial cells (ECs) for transplantation. There are a number of limitations concerning the delivery of therapeutic ECs to assist in the repair of damaged blood vessels, one of them being the availability of appropriate and effective cells for disease treatment. iPS cells, which can give rise to any cell type in the body including ECs (iPS-ECs), may overcome this obstacle and hold great promise regarding the treatment of vascular disease. Indeed, iPS-ECs have shown notable therapeutic potential in pre-clinical studies, which included the ability to incorporate into and re-endothelialize damaged vasculature as well as to inhibit neointimal and inflammatory responses to vascular injury. Even though there are many approaches being researched today aiming towards the advance of the reprogramming methods, many of the cell reprogramming mechanisms underlying the generation of iPS cells and their subsequent differentiation towards various cell lineages still remain relatively unclear. Furthermore, no standardised method exists to generate iPS based on a simple, robust and feeder-free method, with current protocols demonstrating differences in efficiency and population purity. Therefore, to ensure successful translation of iPS cells into effective clinical therapeutics, robust methods and kits for iPS cell generation are required.

Thus, in the present study, human iPS cells were reprogrammed from as little as 1 ml of blood from healthy or diabetic donors based on the robust and highly efficient approach that we have developed, which iPS cells were then differentiated into endothelial cells (iPS-ECs) that displayed typical EC characteristics and which have utility in the prevention and treatment of vascular disease. Thus, our research has for the first time generated human iPS cell from only a few drops of blood in just 5-7 days based on a novel and highly efficient approach which can include use of a substrate-nanoparticle composition in feeder-free conditions.

### SUMMARY OF THE INVENTION

Accordingly, in one aspect, the invention provides a composition suitable for promoting the reprogramming of somatic cells to induced pluripotent stem cells, the composition comprising gelatin and laminin.

Without wishing to be bound by theory, the inventors have found that the efficiency of reprogramming of somatic cells to induced pluripotent stem cells is substantially increased when the somatic cells are grown on a substrate comprising the composition described herein. The composition comprises amounts of gelatin and laminin which have been found by the inventors to provide optimal conditions for reprogramming of somatic cells to induced pluripotent stem cells, which conditions are advantageously feeder-free and xeno-free.

Optionally, the composition comprises gelatin at a concentration of at least about 0.01 w/v %, optionally at least about 0.02 w/v %, optionally at least about 0.03 w/v %, optionally at least about 0.04 w/v %, further optionally at least about 0.05 w/v %.

Optionally, the composition comprises gelatin at a concentration of up to about 10 w/v %, optionally up to about 9 w/v %, optionally up to about 8 w/v %, optionally up to about 7 w/v %, optionally up to about 6 w/v %, optionally up to about 5 w/v %, optionally up to about 4 w/v %, optionally up to about 3 w/v %, optionally up to about 2 w/v %, further optionally up to about 1 w/v %.

Optionally, the composition comprises gelatin at a concentration of about 0.01 to 10 w/v %, optionally about 0.01 to 9 w/v %, optionally about 0.01 to 8 w/v %, optionally about 0.01 to 7 w/v %, optionally about 0.01 to 6 w/v %, optionally about 0.01 to 5 w/v %, optionally about 0.01 to 4 w/v %, optionally about 0.01 to 3 w/v %, optionally about 0.01 to 2 w/v %, optionally about 0.01 to 1 w/v %, further optionally about 1 w/v %.

Optionally, the composition comprises gelatin at a concentration of about 0.02 to 10 w/v %, optionally about 0.02 to 9 w/v %, optionally about 0.02 to 8 w/v %, optionally about 0.02 to 7 w/v %, optionally about 0.02 to 6 w/v %, optionally about 0.02 to 5 w/v %, optionally about 0.02 to 4 w/v %, optionally about 0.02 to 3 w/v %, optionally about 0.02 to 2 w/v %, optionally about 0.02 to 1 w/v %, further optionally about 1 w/v %.

Optionally, the composition comprises gelatin at a concentration of about 0.03 to 10 w/v %, optionally about 0.03 to 9 w/v %, optionally about 0.03 to 8 w/v %, optionally about 0.03 to 7 w/v %, optionally about 0.03 to 6 w/v %, optionally about 0.03 to 5 w/v %, optionally about 0.03 to 4 w/v %, optionally about 0.03 to 3 w/v %, optionally about 0.03 to 2 w/v %, optionally about 0.03 to 1 w/v %, further optionally about 1 w/v %.

Optionally, the composition comprises gelatin at a concentration of about 0.04 to 10 w/v %, optionally about 0.04 to 9 w/v %, optionally about 0.04 to 8 w/v %, optionally about 0.04 to 7 w/v %, optionally about 0.04 to 6 w/v %, optionally about 0.04 to 5 w/v %, optionally about 0.04 to 4 w/v %, optionally about 0.04 to 3 w/v %, optionally about 0.04 to 2 w/v %, optionally about 0.04 to 1 w/v %, further optionally about 1 w/v %.

Optionally, the composition comprises gelatin at a concentration of about 0.05 to 10 w/v %, optionally about 0.05 to 9 w/v %, optionally about 0.05 to 8 w/v %, optionally about 0.05 to 7 w/v %, optionally about 0.05 to 6 w/v %, optionally about 0.05 to 5 w/v %, optionally about 0.05 to 4 w/v %, optionally about 0.05 to 3 w/v %, optionally about 0.05 to 2 w/v %, optionally about 0.05 to 1 w/v %, further optionally about 1 w/v %.

Optionally, the composition comprises laminin at a concentration of at least about 0.1 μg/mL, optionally at least about 1 μg/mL, optionally at least about 5 μg/mL, optionally at least about 10 μg/mL, optionally at least about 15 μg/mL, optionally at least about 20 μg/mL, optionally at least about 25 μg/mL, optionally at least about 30 μg/mL, optionally at least about 35 μg/mL, optionally at least about 40 μg/mL, optionally at least about 45 μg/mL, further optionally at least about 50 μg/mL.

Optionally, the composition comprises laminin at a concentration of up to about 1000 μg/mL, optionally up to about 500 μg/mL, optionally up to about 400 μg/mL, optionally up to about 300 μg/mL, optionally up to about 200 μg/mL, optionally up to about 100 μg/mL, further optionally up to about 50 μg/mL.

Optionally, the composition comprises laminin at a concentration of about 0.1 to 1000 μg/mL, optionally about 1 to 1000 μg/mL, optionally about 5 to 1000 μg/mL, optionally about 10 to 1000 μg/mL, optionally about 15 to 1000 μg/mL, optionally about 20 to 1000 μg/mL, optionally about 25 to 1000 μg/mL, optionally about 30 to 1000 μg/mL, optionally about 35 to 1000 μg/mL, optionally about 40 to 1000 μg/mL, optionally about 45 to 1000 μg/mL, further optionally about 50 to 1000 μg/mL.

Optionally, the composition comprises laminin at a concentration of about 0.1 to 500 μg/mL, optionally about 1 to 500 μg/mL, optionally about 5 to 500 μg/mL, optionally about 10 to 500 μg/mL, optionally about 15 to 500 μg/mL, optionally about 20 to 500 μg/mL, optionally about 25 to 500 μg/mL, optionally about 30 to 500 μg/mL, optionally about 35 to 500 μg/mL, optionally about 40 to 500 μg/mL, optionally about 45 to 500 μg/mL, further optionally about 50 to 500 μg/mL.

Optionally, the composition comprises laminin at a concentration of about 0.1 to 400 μg/mL, optionally about 1 to 400 μg/mL, optionally about 5 to 400 μg/mL, optionally about 10 to 400 μg/mL, optionally about 15 to 400 μg/mL, optionally about 20 to 400 μg/mL, optionally about 25 to 400 μg/mL, optionally about 30 to 400 μg/mL, optionally about 35 to 400 μg/mL, optionally about 40 to 400 μg/mL, optionally about 45 to 400 μg/mL, further optionally about 50 to 400 μg/mL.

Optionally, the composition comprises laminin at a concentration of about 0.1 to 300 μg/mL, optionally about 1 to 300 μg/mL, optionally about 5 to 300 μg/mL, optionally about 10 to 300 μg/mL, optionally about 15 to 300 μg/mL, optionally about 20 to 300 μg/mL, optionally about 25 to 300 μg/mL, optionally about 30 to 300 μg/mL, optionally about 35 to 300 μg/mL, optionally about 40 to 300 μg/mL, optionally about 45 to 300 μg/mL, further optionally about 50 to 300 μg/mL.

Optionally, the composition comprises laminin at a concentration of about 0.1 to 200 μg/mL, optionally about 1 to 200 μg/mL, optionally about 5 to 200 μg/mL, optionally about 10 to 200 μg/mL, optionally about 15 to 200 μg/mL, optionally about 20 to 200 μg/mL, optionally about 25 to 200 μg/mL, optionally about 30 to 200 μg/mL, optionally about 35 to 200 μg/mL, optionally about 40 to 200 μg/mL, optionally about 45 to 200 μg/mL, further optionally about 50 to 200 μg/mL.

Optionally, the composition comprises laminin at a concentration of about 0.1 to 100 μg/mL, optionally about 1 to 100 μg/mL, optionally about 5 to 100 μg/mL, optionally about 10 to 100 μg/mL, optionally about 15 to 100 μg/mL, optionally about 20 to 100 μg/mL, optionally about 25 to 100 μg/mL, optionally about 30 to 100 μg/mL, optionally about 35 to 100 μg/mL, optionally about 40 to 100 μg/mL, optionally about 45 to 100 μg/mL, further optionally about 50 to 100 μg/mL.

Optionally, the laminin is recombinant human laminin. Optionally, the laminin is selected from one or more of laminin 521, laminin 522, laminin 523, laminin 511, laminin 423, laminin 421, laminin 411, laminin 321 (laminin 3A21), laminin 311 (laminin 3A11), laminin 31332, laminin-332 (laminin-3A32), laminin 221, laminin 213, laminin 211, laminin 121, and laminin 111. Optionally, the laminin is selected from one or more of recombinant human laminin 521, recombinant human laminin 522, recombinant human laminin 523, recombinant human laminin 511, recombinant human laminin 423, recombinant human laminin 421, recombinant human laminin 411, recombinant human laminin 321 (laminin 3A21), recombinant human laminin 311 (laminin 3A11), recombinant human laminin 3B32, recombinant human laminin-332 (laminin-3A32), recombinant human laminin 221, recombinant human laminin 213, recombinant human laminin 211, recombinant human laminin 121, and recombinant human laminin 111.

Optionally, the gelatin is recombinant human gelatin.

In particular embodiments, the invention provides a composition suitable for promoting the reprogramming of somatic cells to induced pluripotent stem cells, the composition comprising
gelatin, wherein the gelatin is present at a concentration of at least about 0.01 w/v %, 0.02 w/v %, 0.03 w/v %, 0.04 w/v %, 0.05 w/v %, 0.06 w/v %, 0.07 w/v %, 0.08 w/v %, 0.09 w/v %, 0.1 w/v %, 0.2 w/v %, 0.3 w/v %, 0.4 w/v %, or 0.5 w/v %, and up to a concentration of about 0.8 w/v %, 0.9 w/v %, 1 w/v %, 2 w/v %, 3 w/v %, 4 w/v %, 5 w/v %, 6 w/v %, 7 w/v %, 8 w/v %, 9 w/v %, or 10 w/v %; and laminin, wherein the laminin is present at a concentration of at least about 1 μg/mL, 5 μg/mL, 10 μg/mL, 20 μg/mL, 30 μg/mL, 40 μg/mL, 50 μg/mL. 60 μg/mL. 70 μg/mL. 80 μg/mL. 90 μg/mL. or 100 μg/mL. and up to a concentration of about 100 μg/mL. 200 μg/mL. 300 μg/mL. 400 μg/mL. 500 μg/mL. 600 μg/mL. 700 μg/mL. 800 μg/mL. 900 μg/mL. 1000 μg/mL.

Optionally, the composition comprises
gelatin, wherein the gelatin is present at a concentration of at least about 0.04 w/v %, 0.05 w/v %, 0.06 w/v %, 0.07 w/v %, 0.08 w/v %, 0.09 w/v %, 0.1 w/v %, 0.2 w/v %, 0.3 w/v %, 0.4 w/v %, or 0.5 w/v %, and up to a concentration of about 0.08 w/v %, 0.09 w/v %, 1 w/v %, 2 w/v %, 3 w/v %, 4 w/v %, 5 w/v %, 6 w/v %, 7 w/v %, 8 w/v %, 9 w/v %, or 10 w/v %; and
laminin, wherein the laminin is present at a concentration of at least about 50 μg/mL. 60 μg/mL. 70 μg/mL. 80 μg/mL. 90 μg/mL. or 100 μg/mL. and up to a concentration of about 100 μg/mL. 200 μg/mL. 300 μg/mL. 400 μg/mL. 500 μg/mL. 600 μg/mL. 700 μg/mL. 800 μg/mL. 900 μg/mL. 1000 μg/mL.

Optionally, the composition comprises
gelatin, wherein the gelatin is present at a concentration of at least about 0.04 w/v % and up to a concentration of about 10 w/v %; and
laminin, wherein the laminin is present at a concentration of at least about 50 μg/mL. and up to a concentration of about 200 μg/mL.

Optionally, the composition comprises
gelatin, wherein the gelatin is present at a concentration of at least about 0.04 w/v % and up to a concentration of about 5 w/v %; and
laminin, wherein the laminin is present at a concentration of at least about 50 μg/mL. and up to a concentration of about 200 μg/mL.

Optionally, the composition comprises
gelatin, wherein the gelatin is present at a concentration of at least about 0.04 w/v % and up to a concentration of about 10 w/v %; and
laminin, wherein the laminin is present at a concentration of at least about 50 μg/mL. and up to a concentration of about 100 μg/mL.

Optionally, the composition comprises
gelatin, wherein the gelatin is present at a concentration of at least about 0.04 w/v % and up to a concentration of about 5 w/v %; and
laminin, wherein the laminin is present at a concentration of at least about 50 μg/mL. and up to a concentration of about 100 μg/mL.

Optionally, the composition comprises
gelatin, wherein the gelatin is present at a concentration of at least about 0.5 w/v % and up to a concentration of about 10 w/v %; and
laminin, wherein the laminin is present at a concentration of at least about 50 μg/mL. and up to a concentration of about 200 μg/mL.

Optionally, the composition comprises
gelatin, wherein the gelatin is present at a concentration of at least about 0.5 w/v % and up to a concentration of about 5 w/v %; and
laminin, wherein the laminin is present at a concentration of at least about 50 μg/mL. and up to a concentration of about 200 μg/mL.

Optionally, the composition comprises
gelatin, wherein the gelatin is present at a concentration of at least about 0.5 w/v % and up to a concentration of about 10 w/v %; and
laminin, wherein the laminin is present at a concentration of at least about 50 μg/mL. and up to a concentration of about 100 μg/mL.

Optionally, the composition comprises
gelatin, wherein the gelatin is present at a concentration of at least about 0.5 w/v % and up to a concentration of about 5 w/v %; and
laminin, wherein the laminin is present at a concentration of at least about 50 μg/mL. and up to a concentration of about 100 μg/mL.

Optionally, the composition comprises
gelatin, wherein the gelatin is present at a concentration of at least about 1 w/v % and up to a concentration of about 10 w/v %; and
laminin, wherein the laminin is present at a concentration of at least about 50 μg/mL. and up to a concentration of about 200 μg/mL.

Optionally, the composition comprises
gelatin, wherein the gelatin is present at a concentration of at least about 1 w/v % and up to a concentration of about 5 w/v %; and
laminin, wherein the laminin is present at a concentration of at least about 50 μg/mL. and up to a concentration of about 200 μg/mL.

Optionally, the composition comprises
gelatin, wherein the gelatin is present at a concentration of at least about 1 w/v % and up to a concentration of about 10 w/v %; and
laminin, wherein the laminin is present at a concentration of at least about 50 μg/mL. and up to a concentration of about 100 μg/mL.

Optionally, the composition comprises
gelatin, wherein the gelatin is present at a concentration of at least about 1 w/v % and up to a concentration of about 5 w/v %; and
laminin, wherein the laminin is present at a concentration of at least about 50 μg/mL. and up to a concentration of about 100 μg/mL.

Optionally, the gelatin and laminin, separately or in combination, form at least about 10%, at least about 20%, at least about 30%, at least about 40%, at least about 50%, at least about 60%, at least about 70%, at least about 80%, at least about 90%, at least about 95%, at least about 99%, all or substantially all, of the solids content of the composition. Optionally, the gelatin and laminin, separately or in combination, form at least about 10%, at least about 20%, at least about 30%, at least about 40%, at least about 50%, at least about 60%, at least about 70%, at least about 80%, at least about 90%, at least about 95%, at least about 99%, all or substantially all, of the extracellular matrix content contained in the composition. Optionally, the extracellular matrix content comprises natural and/or synthetic, such as recombinant human, extracellular matrix components,

Optionally, the composition is an aqueous composition. Optionally, the aqueous composition comprises, or consists of, a liquid or a gel. Optionally, the composition is provided as a dry, or substantially dry, composition which may be formed into an aqueous composition by the addition of a solvent. Optionally, the solvent acts to dissolve the solid components, such as the gelatin and/or laminin, of the composition. Optionally, the composition is formed by mixing, optionally dissolving, the gelatin and laminin separately, sequentially, or concurrently in the solvent. Optionally, the composition is formed by mixing, optionally dissolving, the gelatin and laminin separately, sequentially, or concurrently in the solvent so as to form a gel-like composition. By “gel-like”, one understands that the composition has a consistency or viscosity suitable for coating or otherwise applying the composition to a surface such as a surface of a cell culture vessel. Optionally, the solvent acts to dissolve the solid components, such as the gelatin and/or laminin, of the composition. Optionally, the solvent is saline, optionally phosphate buffered saline, or cell culture medium. Optionally, the solvent is water, optionally sterile water. Optionally, the composition is provided as a liquid composition or a gel composition.

Optionally, the composition further comprises one or more additional extracellular matrix components, optionally one or more additional extracellular matrix components. Optionally, the one or more additional extracellular matrix components are selected from collagen, elastin, fibronectin, nidogen, and heparan sulfate proteoglycan. Optionally, or additionally, the composition further comprises Matrigel™, Geltrex™, and/or Cultrex BME™. Optionally, or additionally, the composition further comprises one or more growth factors. Optionally, the one or more growth factors are selected from one or more of transforming growth factor beta (TGF-beta) epidermal growth factor (EGF), insulin-like growth factor (IGF), and fibroblast growth factor (FGF).

Optionally, the composition further comprises a Rho-associated protein kinase (ROCK) inhibitor. Optionally, the ROCK inhibitor is selected from one or more of Y-27632 dihydrochloride (trans-4-[(1R)-1-Aminoethyl]-N-4-pyridinylcyclohexanecarboxamide dihydrochloride), GSK429286A (N-(6-fluoro-1H-indazol-5-yl)-6-methyl-2-oxo-4-[4-(trifluoromethyl)phenyl]-3,4-dihydro-1H-pyridine-5-carboxamide), Y-30141 (4-(1-aminoethyl)-N-(1H-pyrrolo(2,3-b)pyridin-4-yl)cyclohexanecarboxamide dihydrochloride), RKI-1447 (N-[(3-Hydroxyphenyl)methyl]-N′-[4-(4-pyridinyl)-2-thiazolyl]urea dihydrochloride), Fasudil, and Ripasudil (trade name Glanatec).

Optionally, the ROCK inhibitor is present in the composition at a concentration of about 1 μM to 1 mM, optionally about 1 μM to 500 μM, optionally about 1 μM to 100 μM, optionally about 1 μM to 50 μM, optionally about 5 μM to 50 μM, optionally about 10 μM to 50 μM, optionally about 10 μM to 20 μM, further optionally about 10 μM.

Optionally, the composition further comprises genetic elements, optionally episomal genetic elements, which comprise or consist of induced pluripotent stem cells reprogramming factors selected from one or more of Oct4, Sox2, Klf4, c-Myc, Lin28, Nanog and SV40 large T. Optionally, the genetic elements comprise or consist of induced pluripotent stem cells reprogramming factors consisting of Oct4, Sox2, Klf4, c-Myc, Lin28, Nanog and SV40 large T. Optionally, the genetic elements further comprise TERT1.

It will be understood that the genetic elements comprise nucleic acid sequences coding for one or more of the aforementioned reprogramming factors and/or TERT1. Optionally the nucleic acid coding sequences, optionally DNA nucleotide sequences, are comprised in a plasmid or other vector suitable for transfection into somatic cells. It will be understood that the plasmid or other suitable vector is suitable to allow transfection nucleic acid coding sequences into somatic cells contacted with the composition, and optionally, suitable to allow expression of the nucleic acid coding sequences in the somatic cells.

Optionally, the composition further comprises a carrier to which the genetic elements are complexed. Optionally, the carrier is suitable to deliver the genetic elements inside the somatic cells. Optionally, the carrier is selected from one or more of nanoparticles, nanocapsules, micellar systems. Optionally, the carrier comprises nanoparticles for nanoparticle-mediated delivery of the nucleic acid sequences to the somatic cells. It will be understood that the described composition may comprise any type of nanoparticle which is suitable to deliver a load, optionally a load comprising genetic elements, into a somatic cells. Thus, optionally, the nanoparticles comprise lipid-based nanoparticles. Optionally, the lipid-based nanoparticles comprise liposomes, optionally cationic liposomes. Optionally, the nanoparticles comprise inorganic nanoparticles, such as carbon nanotubes, magnetic nanoparticles, calcium phosphate nanoparticles, metal nanoparticles, and quantum dots, optionally nanocrystal quantum dots. Optionally, the metal nanoparticles comprise gold nanoparticles and/or silver nanoparticles. Optionally, the nanoparticles comprise polymer-based nanoparticles, optionally the nanoparticles comprise polymeric nanoparticles. Optionally, the polymer-based nanoparticles comprise or consist of polylactic acid (PLA), poly D,L-glycolide (PLG), polylactide-co-glycolide (PLGA), and/or polycyanoacrylate (PCA). Optionally, the nanoparticles comprise micelles, optionally polymeric micelles. Optionally, the nanoparticles comprise dendrimer nanoparticles. Optionally, the nanoparticles comprise hybrid nanoparticles such liposome-polycation-DNA nanoparticles and multilayered nanoparticles. Optionally, the surface of the nanoparticles comprises anionic functional groups. Optionally, the surface of the biocompatible nanoparticles comprises cationic functional groups.

Optionally, the nanoparticles have a size, optionally average size, in the range of 1 to 1000 nm, optionally 1 to 500 nm, optionally 1 to 400 nm, optionally 1 to 300 nm, optionally 1 to 200 nm, optionally 10 to 200 nm, further optionally 10 to 110 nm. Optionally, said size corresponds to the diameter of the nanoparticle.

Optionally, the nucleic acid sequences are linked to the nanoparticles by mixing the nanoparticles and nucleic acid sequences in serum-free cell culture medium for a time sufficient to allow the nucleic acid sequences to complex with the nanoparticles. Optionally, the nucleic acid sequences are mixed with the nanoparticles in serum-free cell culture medium for at least about 5 minutes, at least about 10 minutes, at least about 20 minutes, at least about 30 minutes. Optionally, the nucleic acid sequences are mixed with the nanoparticles at between about 10 to 30° C., optionally between about 18 to 25° C., between about 20 to 23° C., optionally about room temperature. Optionally, the nucleic acid sequences are mixed with the nanoparticles in serum-free cell culture medium for up to about 120 minutes, optionally up to about 90 minutes, optionally up to about 60 minutes, further optionally up to about 30 minutes. Optionally, the serum-free medium is Opti-MEM™. Optionally, the serum-free medium is Eagle's Minimum Essential Media, buffered with HEPES and sodium bicarbonate, and supplemented with hypoxanthine, thymidine, sodium pyruvate, L-glutamine, trace elements, and growth factors.

As will be understood, the present invention provides a novel substrate comprising laminin and gelatin which reliably facilitates self-renewal of induced pluripotent stem (iPS) cells in a chemically defined, feeder-free and xeno-free stem cell culture system.

Thus, in a further aspect, the invention provides use of the composition described herein for in a method for reprogramming of somatic cells to induced pluripotent stem cells.

In a further aspect, the invention provides a cell culture vessel comprising the composition described herein. The cell culture vessel can be any suitable vessel known in the art for use in cell culture, in particular somatic cell culture and/or stem cell culture. Optionally, the cell culture vessel is selected from one or more of a 96-well plate, 24-well plate, 12-well plate, 6-well plate, T25 flask, T75 flask, T175 flask. Slides, optionally, cell culture slides and cell culture microscope slides, may be considered to be cell culture vessels.

In a further aspect, the invention provides a kit comprising the composition described herein. Optionally, the kit further comprises a cell culture vessel as described herein. Optionally, the kit further comprises a cell culture vessel comprising the composition described herein. Thus, it will be understood that the cell culture vessel comprised in the kit may comprise the composition described herein, that is, the composition described herein may be coated on a surface, optionally a cell growth surface, of the cell culture vessel. Optionally, the kit further comprises instructions for use of the kit. Optionally, the kit comprises the components of the composition described herein, including for example, the gelatin, laminin, carrier and/or ROCK inhibitor, as separate components, or as combinations of components, which may be combined by the end user of the kit. Advantageously, the kit allows the user to simply add somatic cells and a suitable cell culture medium to the substrate and achieve efficient and reliable reprogramming of the somatic cells to induced pluripotent stem cells. The programming may be achieved by introducing genetic elements comprising or consisting of Oct4, Sox2, Klf4, c-Myc, Lin28, Nanog and SV40 large T, and optionally TERT1, into the somatic cells. Introducing the genetic elements can be achieved by standard transfection techniques known in the art. Advantageously, the transfection is achieved using the nanoparticle-mediated delivery of the nucleic acid sequences to the somatic cells as described herein. That is, nanoparticles comprising the nucleic acid sequences and embedded in the composition described herein can deliver the nucleic acid sequences to, and transfect the nucleic acid sequences into, the somatic cells, thus greatly simplifying the reprogramming of the cells. This also reduces the possibility of contamination of the cultured cells since the composition and kit can be produced and provided as sterile products. In addition, reprogramming efficiency can be improved as demonstrated herein.

Thus, in a further aspect, the invention provides a method of reprogramming somatic cells to induced pluripotent stem cells, the method comprising
(i) contacting the somatic cells with the composition described herein;
(ii) introducing genetic elements, optionally episomal genetic elements, that express induced pluripotent stem cells reprogramming factors, and optionally TERT1, into the somatic cells; and
(iii) culturing said expanded somatic cells comprising the genetic elements, thereby producing induced pluripotent stem cells.

Optionally, in step (i), the somatic cells are suspended in cell suspension medium when contacted with the composition. Optionally, the cell suspension medium is a cell culture medium. Optionally, contacting the somatic cells suspended in the suspension medium with the composition causes the suspension medium to dissolve the composition. It will be understood that in compositions comprising nanoparticles and genetic elements, the dissolution of the composition by the suspension medium can improve access between the somatic cells and the nanoparticles and genetic elements.

Optionally, the somatic cells are prepared for producing induced pluripotent stem cells as described hereinbelow. Optionally, the somatic cells are cells as described hereinbelow.

Optionally, the genetic elements that express induced pluripotent stem cells reprogramming factors are introduced into the somatic cells as described hereinbelow. Alternatively, the genetic elements that express induced pluripotent stem cells reprogramming factors are introduced into the somatic cells via the nanoparticles comprised in the composition as described above.

Optionally, the somatic cells comprising the genetic elements are further cultured as described hereinbelow. Optionally, the induced pluripotent stem cells produced from the somatic cells comprising the genetic elements are differentiated into endothelial cells. Optionally, the induced pluripotent stem cells are differentiated into endothelial cells by techniques known in the art, optionally by culturing the induced pluripotent stem cells with growth factors selected from one or more of BMP4, Activin A, CHIR99021, and bFGF2, and optionally VEGF and LY364947.

In another aspect, the invention provides a method of preparing somatic cells for producing induced pluripotent stem cells, the method comprising:
(i) isolating somatic cells from a sample, and
(ii) expanding the somatic cells for a predetermined period of time, wherein the expanded somatic cells express TERT1.

Optionally, the somatic cells are expanded for a predetermined period of time of less than about 14 days, optionally about 13 days, optionally less than about 13 days, optionally about 12 days, optionally less than about 12 days, optionally about 11 days, optionally less than about 11 days, optionally about 10 days, optionally less than about 10 days, optionally about 9 days, optionally less than about 9 days, optionally about 8 days, optionally less than about 8 days, optionally about 7 days, further optionally less than about 7 days.

Optionally, the somatic cells are expanded for a predetermined period of time of at least about 1 day, optionally about 1 day, optionally at least about 2 days, optionally about 2 days, optionally at least about 3 days, optionally about 3 days, optionally at least about 4 days, optionally about 4 days, optionally at least about 5 days, optionally about 5 days, optionally at least about 6 days, optionally about 6 days, optionally at least about 7 days, further optionally about 7 days.

Optionally, the somatic cells are expanded for a predetermined period of time of 2 to 13 days, optionally 2 to 12 days, optionally 2 to 11 days, optionally 2 to 10 days, optionally 2 to 9 days, optionally 2 to 8 days, further optionally 2 to 7 days.

Optionally, the somatic cells are expanded for a predetermined period of time of 3 to 13 days, optionally 3 to 12 days, optionally 3 to 11 days, optionally 3 to 10 days, optionally 3 to 9 days, optionally 3 to 8 days, further optionally 3 to 7 days.

Optionally, the somatic cells are expanded for a predetermined period of time of 4 to 13 days, optionally 4 to 12 days, optionally 4 to 11 days, optionally 4 to 10 days, optionally 4 to 9 days, optionally 4 to 8 days, further optionally 4 to 7 days.

Optionally, the somatic cells are expanded for a predetermined period of time of 5 to 13 days, optionally 5 to 12 days, optionally 5 to 11 days, optionally 5 to 10 days, optionally 5 to 9 days, optionally 5 to 8 days, further optionally 5 to 7 days.

Optionally, the somatic cells are expanded for a predetermined period of time of 6 to 13 days, optionally 6 to 12 days, optionally 6 to 11 days, optionally 6 to 10 days, optionally 6 to 9 days, optionally 6 to 8 days, further optionally 6 to 7 days.

Optionally, the somatic cells are expanded for a predetermined period of time of 7 to 13 days, optionally 7 to 12 days, optionally 7 to 11 days, optionally 7 to 10 days, optionally 7 to 9 days, optionally 7 to 8 days, further optionally about 7 days.

Optionally, TERT1 expression is at least about 10%, optionally at least about 20%, optionally at least about 30%, optionally at least about 40%, optionally at least about 50%, optionally at least about 60%, optionally at least about 70%, optionally at least about 80%, optionally at least about 90%, optionally at least about 95%, optionally about 100%, of the expression of TERT1 in the somatic cells prior to expansion for the predetermined period of time. In other words, TERT1 expression is at least about 10%, 20%, 30%, 40%, 50%, 60%, 70%, 80%, 90%, 95% or 100%, of the expression of TERT1 in unexpanded somatic cells, i.e. the isolated somatic cells prior to undergoing the expansion step (ii) noted above.

Without wishing to be bound by theory, the present inventors have discovered that TERT1 expression decreases during expansion of somatic cells and that TERT1 expression is required for reprogramming of the somatic cells to iPS cells. Therefore, the expanded somatic cells produced according to the methods described herein express TERT1 such that the expanded cells are suitable for producing induced pluripotent stem cells.

Optionally, the TERT1 expression is measured in expanded and/or unexpanded somatic cells by measuring TERT1 mRNA levels and/or TERT1 protein levels. Optionally, the TERT1 expression in expanded cells is compared to TERT1 expression in unexpanded cells to determine the relative expression levels of TERT1. In other words, the TERT1 expression in expanded cells is normalised relative to the TERT1 expression in unexpanded cells to indicate the relative expression levels of TERT1. Optionally, the TERT1 mRNA expression is measured by real time polymerase chain reaction, optionally by extracting RNA from the somatic cells, reverse transcribing the mRNA to cDNA, and performing real time polymerase chain reaction using TERT1 primers. Optionally, the TERT1 protein expression is measured by using protein extracted from the somatic cells, optionally western blotting the protein extracts using a TERT1 specific antibody to detect TERT1 expression. Optionally, the TERT1 protein expression is measured by using a TERT1 specific antibody comprising a fluorophore to bind TERT1 in somatic cells somatic cells and to observe said binding under a fluorescence microscope in order to determine the expression levels of TERT1. TERT1 specific antibodies are well known in the art, such as Anti-Telomerase reverse transcriptase antibody [Y182] (ab32020). Fluorophores are well known in the art and include fluorescein, Cy5, etc. It will be understood that methods of detecting gene expression at mRNA or protein level are known in the art and can be employed to measure TERT1 expression as described herein.

Optionally, the method of preparing somatic cells for producing induced pluripotent stem cells further comprises the step of measuring TERT1 expression in the expanded and/or unexpanded somatic cells. Optionally, the method of preparing somatic cells for producing induced pluripotent stem cells further comprises measuring TERT1 expression in the expanded and unexpanded somatic cells and determining the relative expression level of TERT1. Optionally, the method of preparing somatic cells for producing induced pluripotent stem cells further comprises the step of measuring TERT1 expression in the expanded and/or unexpanded somatic cells to determine the suitability of the expanded cells for producing the induced pluripotent stem cells, wherein the expanded somatic cells are determined to be suitable for producing the induced pluripotent stem cells if the expanded somatic cells express TERT1. Optionally, the expanded somatic cells are determined to be suitable for producing the induced pluripotent stem cells if the TERT1 expression is at least about 10%, optionally at least about 20%, optionally at least about 30%, optionally at least about 40%, optionally at least about 50%, optionally at least about 60%, optionally at least about 70%, optionally at least about 80%, optionally at least about 90%, optionally at least about 95%, optionally about 100%, of the expression of TERT1 in the somatic cells prior to expansion, optionally prior to expansion for a predetermined period of time as described herein. In other words, TERT1 expression is at least about 10%, 20%, 30%, 40%, 50%, 60%, 70%, 80%, 90%, 95% or 100%, of the expression of TERT1 in unexpanded somatic cells, e.g. the isolated somatic cells prior to undergoing the expansion step (ii) noted above.

Optionally, the somatic cells are mammalian cells, further optionally human cells. Optionally, the somatic cells are primary cells or immortalized cells. Optionally, the somatic cells are isolated from a biological sample obtained from a subject, optionally a human subject. Optionally, the biological sample is a sample of tissue, optionally human tissue. Optionally, the tissue is selected from one or more of blood, skin, lung, pancreas, liver, stomach, intestine, heart, reproductive organ, bladder, kidney, urethra and other urinary organ tissue. Optionally, the somatic cells are selected from peripheral blood mononuclear cells such as monocytes and lymphocytes (natural killer (NK), B and T lymphocytes), erythrocytes such as neutrophils, basophils and eosinophils, macrophages, sertoli cells, endothelial cells, granulosa epithelial, neurons, pancreatic islet cells, epidermal cells, epithelial cells, hepatocytes, hair follicle cells, keratinocytes, adipocytes, hematopoietic cells, melanocytes, chondrocytes, fibroblasts, and muscle cells such as cardiac muscle cells. Optionally, the somatic cells comprise adult stem cells such as hematopoietic stem cells, neural stem cells, and mesenchymal stem cells.

Optionally, the sample is a blood sample and the volume of said blood sample is less than about 10 ml, optionally less than about 5 ml, optionally less than about 2.5 ml, optionally less than about 1 ml, further optionally about 1 ml.

Optionally, the subject is a human subject, and said subject suffers from diabetes. Optionally, said diabetes is type 1 diabetes, type 2 diabetes, or gestational diabetes.

Optionally, the peripheral blood mononuclear cells have not been mobilized prior to obtaining the sample from the subject. Further optimally the peripheral blood mononuclear cells have not been mobilized with extrinsically applied granulocyte colony stimulating factor (G-CSF) or granulocyte macrophage colony-stimulating factor (GM-CSF) prior to obtaining the sample from the subject.

Optionally, the somatic cells are expanded in a suitable expansion medium, wherein optionally the expansion medium comprises serum free medium (SFM) supplemented with one or more growth factors, wherein, optionally, said growth factors are selected from erythropoietin (EPO), IL-3, stem cell factor (SCF), insulin-like growth factor-1 (IGF-1), dexamethasone, and holo-transferrin.

Optionally, the isolated somatic cells are expanded in the expansion medium for a first expansion period of about two to four days, optionally about two to three days, further optionally about three days. Optionally, the isolated somatic cells are plated at a density of about 2-6×10⁶ cells per ml, about 3-5×10⁶ cells per ml, about 4×10⁶ cells per ml, expanded in the expansion medium for a first expansion period of about two to four days, optionally about two to three days, further optionally about three days. Optionally, the somatic cells expanded in the first expansion period are further expanded for a second expansion period of about two to four days, optionally about two to three days, further optionally about three days. Optionally, the somatic cells expanded in the first expansion period are subsequently plated at a density of about 0.5-2×10⁶ cells per ml, about 0.5-1.5×10⁶ cells per ml, about 1×10⁶ cells per ml, and expanded in the expansion medium for a second expansion period of about two to four days, optionally about two to three days, further optionally about three days.

Optionally, the method further comprises (iii) cryopreserving the expanded somatic cells. Optionally, the method further comprises (iii) cryopreserving the expanded somatic cells in freezing medium, optionally wherein the freezing medium comprises about 50% foetal bovine serum (FBS), about 40% serum free medium (SFM) and about 10% dimethyl sulfoxide (DMSO).

In a further aspect, the present invention provides a method for determining the suitability of expanded somatic cells for producing induced pluripotent stem cells, the method comprising
(i) measuring the expression of TERT1 in the expanded somatic cells; and
(ii) determining the suitability of the expanded somatic cells for producing the induced pluripotent stem cells based on the measured expression of TERT1.

Optionally, the expanded somatic cells are determined to be suitable for producing the induced pluripotent stem cells if the expanded somatic cells express TERT1. Optionally, the expanded somatic cells are determined to be suitable for producing the induced pluripotent stem cells if the TERT1 expression is at least about 10%, optionally at least about 20%, optionally at least about 30%, optionally at least about 40%, optionally at least about 50%, optionally at least about 60%, optionally at least about 70%, optionally at least about 80%, optionally at least about 90%, optionally at least about 95%, optionally about 100%, of the expression of TERT1 in the somatic cells prior to expansion, optionally prior to expansion for a predetermined period of time as described herein. In other words, TERT1 expression is at least about 10%, 20%, 30%, 40%, 50%, 60%, 70%, 80%, 90%, 95% or 100%, of the expression of TERT1 in unexpanded somatic cells, e.g. the isolated somatic cells prior to undergoing the expansion step (ii) noted above.

Optionally, the TERT1 expression is measured in expanded and/or unexpanded somatic cells by measuring TERT1 mRNA levels and/or TERT1 protein levels, optionally by measuring TERT1 mRNA levels and/or TERT1 protein levels as described herein.

Optionally, the somatic cells are expanded for a predetermined period of time as described herein.

Optionally, the somatic cells are selected from the somatic cells described herein.

In a further aspect, the present invention provides a method for producing induced pluripotent stem cells, the method comprising:
(a) introducing genetic elements, optionally episomal genetic elements, that express induced pluripotent stem cells reprogramming factors into expanded somatic cells produced according to the method of preparing somatic cells described herein, and
(b) culturing said expanded somatic cells comprising the genetic elements, thereby producing induced pluripotent stem cells.

Optionally, the genetic elements are introduced into the expanded somatic cells via a non-viral transfection method, optionally via electroporation. Optionally, the genetic elements are introduced into the expanded somatic cells using a lipid-based transfection reagent, optionally wherein said lipid-based transfection reagent is Endofectin™.

Optionally, the genetic elements comprise or consist of induced pluripotent stem cells reprogramming factors selected from one or more of Oct4, Sox2, Klf4, c-Myc, Lin28, Nanog and SV40 large T. Optionally, the genetic elements comprise or consist of induced pluripotent stem cells reprogramming factors consisting of Oct4, Sox2, Klf4, c-Myc, Lin28, Nanog and SV40 large T.

Optionally, in step (b), the expanded somatic cells comprising the genetic elements are cultured in expansion medium, wherein optionally the expansion medium comprises serum free medium (SFM) supplemented with one or more growth factors, wherein, optionally, said growth factors are selected from erythropoietin (EPO), IL-3, stem cell factor (SCF), insulin-like growth factor-1 (IGF-1), dexamethasone, and holo-transferrin. Optionally, the expanded somatic cells comprising the genetic elements are cultured in expansion medium for at least about 1-3 days, optionally about 1-3 days, further optionally about 2 days. Optionally, the expanded somatic cells comprising the genetic elements are plated at a density of about 1-3×10⁶ cells per 3.8 cm² of a cell growth surface, 35 optionally about 2×10⁶ cells per 3.8 cm² of a cell growth surface, and cultured in expansion medium for at least about 1-3 days, optionally about 1-3 days, further optionally about 2 days. It will be understood that a cell growth surface typically comprises the base, or the base of a well, of a cell culture plate suitable for cell adhesion and growth.

Optionally, following the culturing in expansion medium, the expanded somatic cells comprising the genetic elements are seeded onto inactivated mouse embryonic fibroblasts (MEFs) and cultured in reprogramming medium. Optionally, the reprogramming medium is medium suitable to allow the expanded somatic cells comprising the genetic elements to become reprogrammed into pluripotent stem cells. Optionally, the reprogramming medium comprises serum free medium (SFM, such as knockout-DMEM) supplemented with one or more of: a serum replacement composition (such as Knockout Serum Replacement) comprising amino acids (such as Glycine, L-histidine, L-isoleucine, L-methionine, L-phenylalanine, L-proline, L-hydroxyproline, L-serine, L-threonine, L-tryptophan, L-tyrosine, L-valine), vitamins and/or antioxidants (such as thiamine, reduced glutathione, ascorbic acid 2-PO₄), trace elements (such as Ag⁺, Al³⁺, Ba²⁺, Cd²⁺, Co²⁺, Cr³⁺, Ge⁴⁺, Se⁴⁺, Br⁻, I⁻, F⁻, Mn²⁺, Si⁴⁺, V⁵⁺, MO⁶⁺, Ni²⁺, Rb⁺, Sn²⁺, Zr⁴⁺) and/or proteins (such as transferrin (iron-saturated), insulin, lipid-rich albumin), as well as bFGF, β-mercaptoethanol and MEM (minimum essential medium) non essential amino acids (MEM NEAA). Optionally, following the culturing in expansion medium, the expanded somatic cells comprising the genetic elements are seeded onto inactivated mouse embryonic fibroblasts (MEFs) and cultured in reprogramming medium for at least about 1-2 days, optionally about 1-2 days, further optionally for about 1 day. Optionally, following the culturing in expansion medium, the expanded somatic cells comprising the genetic elements are seeded onto inactivated mouse embryonic fibroblasts (MEFs) and cultured in reprogramming medium at a density of 1×10⁴ to 1×10⁶ cells, optionally 8×10⁴ to 1×10⁵ cells, per 3.8 cm² of a cell growth surface for at least about 1-2 days, optionally about 1-2 days, further optionally for about 1 day.

Optionally, following culturing on the inactivated mouse embryonic fibroblasts (MEFs), the expanded somatic cells comprising the genetic elements are removed from the MEFs and cultured in reprogramming medium comprising sodium borate. Optionally, following culturing on the inactivated mouse embryonic fibroblasts (MEFs), the expanded somatic cells comprising the genetic elements are removed from the MEFs and cultured in reprogramming medium comprising sodium borate for at least about 1-2 days, optionally about 1-2 days, further optionally for about 1 day. Optionally, the sodium borate is present in the reprogramming medium at a concentration of about 0.025 to 2.5 mM, optionally about 0.1 to 1 mM, further optionally about 0.25 mM.

Optionally, the reprogramming medium comprising sodium borate is replaced with fresh reprogramming medium comprising sodium borate every day. Optionally the reprogramming medium comprising sodium borate is replaced with fresh reprogramming medium comprising sodium borate every day for about 4-8 days, optionally about 5-7 days, further optionally about 6 days.

Optionally, the reprogramming medium comprising sodium borate is replaced with conditioned medium comprising sodium borate and basic fibroblast growth factor every day until one or more cell colonies comprising induced pluripotent stem cells are formed. Optionally, the basic fibroblast growth factor the sodium borate is present in the reprogramming medium at a concentration of about 0.1 ng/ml to 1 μg/ml, optionally about 0.1 ng/ml to 1 μg/ml, further optionally about 10 ng/ml, and the sodium borate is present in the reprogramming medium at a concentration of about 0.025 to 2.5 mM, optionally about 0.1 to 1 mM, further optionally about 0.25 mM.

In a further aspect, the present invention provides an expanded somatic cell produced according to the method of preparing somatic cells for producing induced pluripotent stem cells described herein.

In a further aspect, the present invention provides an expanded somatic cell expressing TERT1. Optionally, in the expanded somatic cell expressing TERT1, TERT1 expression is at least about 10%, optionally at least about 20%, optionally at least about 30%, optionally at least about 40%, optionally at least about 50%, optionally at least about 60%, optionally at least about 70%, optionally at least about 80%, optionally at least about 90%, optionally about 100%, of the expression of TERT1 in the unexpanded somatic cells. Optionally, the expanded somatic cell expressing TERT1 is produced according to the method of preparing somatic cells for producing induced pluripotent stem cells described herein.

Optionally, the TERT1 expression is measured in expanded and/or unexpanded somatic cells by measuring TERT1 mRNA levels and/or TERT1 protein levels. Optionally, the TERT1 mRNA expression in expanded cells is compared to TERT1 mRNA expression in unexpanded cells to determine the relative expression levels of the TERT1 mRNA levels. In other words, the TERT1 mRNA expression in expanded cells is normalised relative corresponds to the TERT1 mRNA expression normalised relative to TERT1 mRNA expression in unexpanded cells to indicate the relative expression levels of TERT1. Optionally, the TERT1 mRNA expression is measured by real time polymerase chain reaction, optionally by extracting RNA from the somatic cells, reverse transcribing the mRNA to cDNA, and performing real time polymerase chain reaction using TERT1 primers. Optionally, the TERT1 protein expression is measured by using protein extracted from the somatic cells, optionally western blotting the protein extracts using a TERT1 specific antibody to detect TERT1 expression. Optionally, the TERT1 protein expression is measured by using a TERT1 specific antibody comprising a fluorophore to bind TERT 1 in somatic cells somatic cells and to observe said binding under a fluorescence microscope in order to determine the expression levels of TERT1. TERT1 specific antibodies are well known in the art, such as Anti-Telomerase reverse transcriptase antibody [Y182] (ab32020). Fluorophores are well known in the art and include fluorescein, Cy5, etc. It will be understood that methods of detecting gene expression at mRNA or protein level are known in the art and can be employed to measure TERT1 expression as described herein.

In a further aspect, the present invention provides an induced pluripotent stem cell produced according to the method for producing induced pluripotent stem cells described herein.

In a further aspect, the present invention provides an induced pluripotent stem cell produced from the expanded somatic cell described herein. Optionally, the induced pluripotent stem cell is produced according to the method for producing induced pluripotent stem cells described herein.

In a further aspect, the present invention provides an induced pluripotent stem cell described herein for use in therapy. Optionally, the induced pluripotent stem cell described herein is for use in the treatment of, for example, Alzheimer's disease, Parkinson's disease, cardiovascular disease, diabetes, diabetic complications, heart failure, kidney and lives diseases, cancer, amyotrophic lateral sclerosis, or genetic conditions such as Fanconi anemia or cystic fibrosis. It will be understood that the induced pluripotent stem cell described herein may be employed in treating other conditions and diseases amenable to stem cell therapy.

In a further aspect, the present invention provides an expanded somatic cell described herein and/or induced pluripotent stem cell described herein for use in research, optionally experimental research.

As will be understood from the above description of features of aspects of the invention, optional features may be combined, even if not explicitly stated, in any combination. Optional features have been recited as such for convenience and brevity, but in no way limit the combination of features in the various aspects of the described invention. The combination of features is only limited by the chemical, physical or structural incompatibilities, and suitability to achieve the aim of the invention, the determination of which is well within the knowledge and abilities of the skilled reader based on the present disclosure.

By “expand”, “expansion”, and the like, as used herein, it is meant that isolated somatic cells are allowed to grow and replicate under controlled conditions to increase the number of cells in accordance with the usual meaning of the word in the field of cell culture. It will be understood that the period of time for which somatic cells may be expanded is calculated from the moment the somatic cells are plated or otherwise allowed to expand in culture, which may occur immediately following their isolation from a sample, or following a period of storage, such as frozen storage, before the isolated cells are plated or otherwise allowed to expand in culture. The end of the expansion period occurs when the expanded somatic cells are harvested, or otherwise collected, for subsequent storage or for production of induced pluripotent stem cells therefrom.

By “culture”, “culturing”, and the like, as used herein, it is meant that isolated somatic cells comprising the genetic elements, optionally episomal genetic elements, are allowed to grow, and optionally replicate, under controlled conditions, e.g. in appropriate medium, atmosphere and temperature, in accordance with the usual meaning of the word in the field of cell culture.

By “about”, as used herein, it is meant that the recited value may be precisely the recited value, optionally ±5% of the recited value, optionally ±10% of the recited value, optionally ±15% of the recited value, optionally ±20% of the recited value, optionally ±30% of the recited value, optionally ±40% of the recited value, further optionally ±50% of the recited value. When used in relation to a specified number of days, by “about”, as used herein, it is meant that the recited number of days may be precisely the recited value, optionally ±3 days, optionally ±2 days, optionally ±1 day, optionally ±18 hours, optionally ±12 hours, optionally ±6 hours, further optionally ±3 hours.

By “comprise”, it is understood that the disclosed feature may alternatively consist of, or consist essentially of, the described component(s) of the feature.

### BRIEF DESCRIPTION OF THE DRAWINGS

The embodiments of the present invention will now be described, by way of example, with reference to the accompanying drawings, in which:

FIG. 1 demonstrates TERT1 to be a key mediator of iPS cells reprogramming. (A) The expression levels of TERT1 are unexpectedly abolished after 14 days of culture of the mononuclear cells (MNCs) obtained from the health volunteers and diabetic patients. (B) When TERT1 was knocked down in 7 days MNCs by shRNA, the reprogramming efficiency was reduced to 0% indicating that TERT1 expression is an important mediator of iPS cells generation. (C) Control MNCs with normal TERT1 levels generated iPS cells colonies which express pluripotency stem cell markers. Based on these findings, a new protocol of generating pluripotent stem cells, as described herein, has been developed. MNCs obtained from only few drops of blood may be expanded for only 7 days and subjected to iPS reprogramming. This protocol generates iPS cells colonies within one week in a reproducible and highly efficient manner. The establishment of such novel and short protocols are powerful tools for personalised and regenerative medicine.

FIG. 2 depicts generation and characterisation of iPS cells obtained from a few drops of blood based on the presently disclosed novel approach. (A) Diagram explaining the iPSC generation from a few drops of blood samples from health volunteers and diabetic patients. (B) iPSCs form round colonies with defined limits. (C) Immunofluorescence assays show that iPSCs express pluripotent markers such as CDy1 (in vitro live staining), Oct4, TRA-1-60 and Lin28. (D) iPSCs express Oct4, Lin28 and Nanog at an mRNA level. (E) iPSCs express TRA-1-60, Oct4 and Lin28 at a protein level. (F) iPSCs form teratomas in vivo. These data confirm that these cells are indeed pluripotent stem cells.

FIG. 3 depicts the differentiation of the iPS cells towards functional endothelial cells. (A) Diagram of iPS cell differentiation towards endothelial cell (EC) lineage. (B) iPS-ECs morphology. (C) qPCR data showing iPS-ECs express endothelial markers at a mRNA level. (D) When differentiated, iPS-ECs express VE-cadherin at a protein level and stop expressing pluripotent marker OCT4. (E) iPS-ECs form tight junctions in vitro and express VE-cadherin, PECAM-1 and ZO-1. (F) iPS-ECs take up LDL and they form vascular structures in in vitro Tube Formation Assays. These data confirm that these cells are functional ECs and are powerful tools for drug screening and cell based therapies.

FIG. 4 depicts the results of a screening assay to profile the expression levels of various factors potentially involved in the generation of iPS cells from monocular cells. Expression levels in the monocular cells on days 9 and 14 post-expansion was measured.

FIG. 5 depicts (A) iPS cells generated from expanded monocular cells transfected with the non-integrating episomal plasmid vectors pEB-05 (overexpressing Oct4, Sox2, Klf4, c-Myc and Lin28), and pEB-Tg vector (overexpressing SV40 large T antigen), supplemented by TERT1, and grown on a novel substrate. Typical iPS cell colonies with well-defined round limits are observed. (B) depicts the iPS cell colonies stained positive for the pluripotent marker CDy1.

### DETAILED DESCRIPTION

Materials & Methods

Blood Mononuclear Cells (MNCs) Isolation and Expansion

1 to 20 ml of non-mobilised peripheral blood were collected by venepuncture in EDTA-coated 4 ml tubes. The blood was separated by gradient centrifugation by layering it on Histopaque solution (1:1 ratio) and spinning for 30 minutes at 550 g at room temperature. The Mononuclear cells (MNCs) form a buffy coat between the plasma layer and the Histopaque buffer layer. The supernatant was discarded and the cells resuspended in 1 ml of MNC medium, which medium comprises serum free medium (SFM) supplemented with erythropoietin (EPO), IL-3, stem cell factor (SCF), insulin-like growth factor-1 (IGF-1), dexamethasone, and holo-transferrin. Specifically, the SFM (for 100 ml) comprises: 49 ml IMDM (Life Technologies 21056023), 49 ml F12 Nutrient Mix (Life Technologies 21765029), 1 ml ITS-X (Life Technologies 41400045), 1 ml Chemically defined lipid concentrate (Life Technologies 11905031), 1 ml Penicillin/Streptavidin, 1 ml Glutamax, 5 mg Ascorbic Acid (SIGMA A8960), 0.5 g BSA (SIGMA A9418), 1.8 μl 1-Thioglycerol (SIGMA M6145), and the MNC comprises SFM supplemented with: 2 U ml Recombinant human erythropoietin (EPO; R&D Systems, cat. no. 287-TC-500), 10 ng/ml IL-3 (IL-3; PeproTech, cat no. 200-03), 100 ng/ml Recombinant human stem cell factor (SCF; PeproTech, cat. no. 300-07), 40 ng/ml Recombinant human insulin-like growth factor-1 (IGF-1; PeproTech, cat. no. 100-11), 1 μM dexamethasone (Sigma-Aldrich, cat. no. D2915), and 100 μg/ml Human holo-transferrin (R&D Systems, cat. no. 2914-HT-100MG). The cells were counted and plated at a density of −4 million cells per ml in 12-well (1 ml per well) or 6-well (1-4 mls per well) plates. On day 3 after plating, the medium was changed by collecting all cells and spinning for 5 minutes at 250 g. The cells were cryopreserved from day 7 in freezing medium (50% FBS, 40% SFM and 10% DMSO) or used for reprogramming straight away.

Reprogramming

2 million cells were transfected with 10 μg of plasmid (8 μg of pEB-05 and 2 μg of pEB-antigen T) via 35 electroporation using the Lonza CD34 nucleofector kit and Amaxa nucleofector (program T-016). After the electroporation, the cells were plated in 2 ml of MNC medium in a well of a 12-well plate, i.e. 2 million cells per well. On day 2 after transfection, the cells were collected, counted and seeded onto inactivated mouse embryonic fibroblasts (MEFs) in reprogramming medium at a density of 100,000-80,000 cells per well of a 12-well plate (i.e. each well has a growth surface are of 40 approximately 3.8 cm²). The reprogramming media comprises Knockout DMEM (Invitrogen, SKU-10829-018), 20% Knockout Serum Replacement (Invitrogen SKU 10828-028), 10 ng/ml basic fibroblast growth factor (bFGF Miltenyi Biotec, 130-093-837), 0.1 mM β-mercaptoethanol and 0.1 mM MEM non essential amino acids (MEM NEAA). On day 3 after transfection, the medium was collected in tubes and centrifuged for 5 minutes at 300 g. The pellets were resuspended with the remaining volume of new medium and plated back into their wells. Sodium borate (NaB) was added at a concentration of 0.25 mM to the medium until colonies were picked. The reprogramming medium (with NaB) was changed every day with fresh reprogramming medium (with NaB). From day 9 after transfection and thereafter, the reprogramming medium (with NaB) was replaced with conditioned medium with FGF2 (10 ng/ml) and NaB (0.25 mM). The medium was changed every day, i.e. with fresh conditioned medium with FGF2 (10 ng/ml) and NaB (0.25 mM). Colonies appeared from 10 day 7-10. Once the colonies were picked, cell lines were established and cultured in reprogramming medium supplemented with FGF2 [at 10 ng/ml].

In a further development of our method, mononuclear blood cells (MNC)s obtained from healthy donor and expanded, as described above, for about 7 days, were defrosted in MNC medium according to standard defrosting protocols. The wells of 6-well plates were coated with different substrates (see Table 4) overnight at 4° C. Each well of a 6-well plate typically has a growth surface area of approximately 9.5 cm². The substrates were: a novel substrate of the present invention comprising gelatin 1%, laminin 50 μg/mL, formulated to a thick gel-like solution by addition of phosphate buffered saline (PBS) and mixing the gelatin and laminin in the PBS (ES) Matrigel, Gelatin with Matrigel™, Cell Matrix (CellMatrix Basement Membrane Gel (ATCC® ACS-3035™), Matrigel™ with Cell Matrix (CellMatrix Basement Membrane Gel (ATCC® ACS-3035™), and Matrigel™. Next day, 2,000,000 MNCs were transfected, as described above, using the non-integrating episomal plasmid vectors pEB-05 (overexpressing Oct4, Sox2, Klf4, c-Myc and Lin28), and pEB-Tg vector (overexpressing SV40 large T antigen) (Chou et al. [5] and Dowey et al. [6]), optionally supplemented by TERT1. TERT1 was cloned to the vector (cloning sequence obtained from NM_198253.2), generating a plasmid which overexpresses TERT1, using standard cloning techniques known in the art. The 2,000,000 transfected MNCs were added into each well of a 6-well plate in 2 ml MNC medium. MNC medium and ReproTeSR™ medium were added according to manufacturer's instructions. ReproTeSR™ (Stem Cell Technologies) is a complete, defined, serum-free and xeno-free reprogramming medium. This medium is used during the generation of iPS cells from somatic cells, such as fibroblasts and other cell types, under feeder-free conditions, and according to manufacturer's instructions (which are available at https://cdn.stemcell.com/media/files/pis/DX20217-PIS_1_3_0. pdf?_ga=2.216708116.343397011.15 27158806-1504615269.1527158806). Rho-associated protein kinase (ROCK) inhibitor was added on day 3. The ROCK inhibitor, Y-27632 dihydrochloride (trans-4-[(1R)-1-Aminoethyl]-N-4-pyridinylcyclohexanecarboxamide dihydrochloride), was obtained from TOCRIS (Cat. No. 1254). Stock solution of 10 mM was further diluted during use to 1:1000. Colonies were observed from day 5. From day 5, the ReproTeSR medium was changed daily (2 ml) supplemented with ROCK Inhibitor (10 mM stock solution was diluted during use to 1:1000), and colonies were picked, expanded, characterised and frozen down according to standard protocols described herein and/or known in the art.

Again, in a further development of this method, the reprogramming factors were introduced into the MNCs via nanoparticles. That is, nanoparticles from a 2 mM stock were diluted 1:2 in 5% dextrose to obtain a 1 mM solution. The correct volumes for the nanoparticles and for the plasmids is determined based on the cell number used. Good results have been obtained by using 1.5 μl of a 1 mM nanoparticle solution with 0.25 μg of plasmid DNA (about 1 μg of DNA is suitable for transfecting about 1×10⁶ cells). First, serum-free Optimem was added to the required volume of DNA (reprogramming plasmids) up to 125 μL (for one well of a 6-well plate). Serum-free Optimem was also added to the volume of nanoparticles up to 125 μL. Then, the 125 μL of DNA-Optimem is added dropwise to the 125 μL nanoparticles-Optimem. The DNA-nanoparticle mixture is incubated at room temperature for 30 minutes. Then, the DNA-nanoparticle mixture is added to the substrate, thus producing a product comprising a substrate embedded with reprogramming factors comprised on nanoparticles.

As will be understood, DNA is, itself, a polyelectrolyte—the negatively charged sugar phosphate backbone of DNA influences, among other things: (i) the conformation and dynamics of DNA in solution, (ii) the nature of its chemical and physical interactions with both small and large molecules, and (iii) the manner in which it adsorbs at surfaces and interfaces. Owing to the above considerations, many approaches to the delivery of DNA have focused on the design of positively charged polymers. Cationic polymers can interact with DNA in solution through electrostatic interactions to form aggregates or assemblies with sizes, charges, and other properties that can promote the internalization and processing of DNA by cells.

The substrate may further comprise ROCK inhibitor as described herein, e.g. Y-27632 dihydrochloride, at a concentration of approximately 10-20 μM.

### Teratoma Formation Assay

iPS cells (1×10⁶) were mixed with Matrigel and subcutaneously injected into severe combined immunodeficiency (SCID) mice. Eight weeks later, the plugs were harvested, sectioned for HE staining and teratoma formation observed.

Cell Differentiation

Human induced pluripotent stem (iPS) cells cultured under feeder-free conditions were detached using dissociation medium and seeded on mouse collagen IV (BD mouse collagen IV-5 μg/ml)-35 coated plates in EGM-2 media (Lonza) plus 10% FBS. The dissolution medium comprises a reagent to dissociate the human iPS colonies into single cells (RCHETP002, Reinnervate). The medium was supplemented with 25 ng/ml BMP4, 12 ng/ml Activin A, 8 μM CHIR99021, and 20 ng/ml FGF2 (bFGF Miltenyi Biotec, 130-093-837) (day 0 of differentiation). After 48 hours (day 2), the medium was replaced with EGM-2 plus 10% FBS supplemented with 50 ng/ml VEGF, 10 ng/ml FGF2 and 10 μM LY364947 (Sigma) and refreshed every other day. On day 6 of differentiation, MACS-mediated selection for CD144-expressing cells was performed and the selected cells were seeded on mouse collagen IV-coated plates. MACS® Technology utilises microbead technology to isolate any cell type from a mixed population of cells by magnetically labelling cells of interest in a sample with MACS MicroBeads, applying the sample to a MACS Column placed in a MACS Separator, and capturing and then collecting the magnetically labeled cells on the column. The medium used was EGM-2 with 10% FBS supplemented with 50 ng/ml VEGF and 10 μM LY364947.

RNA Extraction, Reverse Transcriptase-Polymerase Chain Reaction (RT-PCR) and Real-Time PCR

Cells were harvested and washed with cold PBS, lysed with Qiazol and the RNA was purified using the RNeasy mini kit (Qiagen) according to the manufacturer's instructions. RNA yield was determined using the NanoDrop spectrophotometer (NanoDrop Technologies). Total RNA (2 μg) was converted to cDNA. Quantitative PCR (qPCR) was done using SYBR Green (Life Technologies) and detection was achieved using the thermocycler LightCycler 480 sequence detector (Roche). Primer sequences are listed in Table 1. Expression of target genes was normalized to reference gene GAPDH.

TABLE 1
Primer sequences.

| | | | | SEQ |
|---|---|---|---|---|
| | | SEQ | | ID |
| Gene | Forward | ID NO. | Reverse | NO. |
| Oct4 | AGAACATGTGTAAGCTGCGG | 1 | GTTGCCTCTCACTCGGTTC | 2 |
| Lin28 | GCAGAAGCGCAGATCAAAAG | 3 | CGGACATGAGGCTACCATATG | 4 |
| Nanog | GAAATACCTCAGCCTCCAGC | 5 | GCGTCACACCATTGCTATTC | 6 |
| KDR | ATAGAAGGTGCCCAGGAAAA | 7 | GTCTTCAGTTCCCCTCCATTG | 8 |
| | G | | | |
| CD144 | AAACACCTCACTTCCCCATC | 9 | ACCTTGCCCACATATTCTCC | 10 |
| eNOS | GGTACATGAGCACTGAGATC | 11 | GCCACGTTGATTTCCACTG | 12 |
| | G | | | |
| TERT1 | GCACGGCTTTTGTTCAGATG | 13 | CGGTTGAAGGTGAGACTGG | 14 |
| Cbp/300 | ATGGACGCCGAACTCATC | 15 | CCAAGTCCGAGAAGCAGTC | 16 |
| CITED4 | | | | |
| SOX18 | TCATGGTGTGGGCAAAGG | 17 | CGTTCAGCTCCTTCCACG | 18 |
| SOX17 | AGAATCCAGACCTGCACAAC | 19 | GCCGGTACTTGTAGTTGGG | 20 |
| TCF3 | GAGAAGCCCCAGACCAAAC | 21 | ACCACACCTGACACCTTTTC | 22 |
| FOXC1 | AGTAGCTGTCAAATGGCCTTC | 23 | TTAGTTCGGCTTTGAGGGTG | 24 |
| mTOR | CAAGAACTCGCTGATCCAAAT | 25 | GCTGTACGTTCCTTCTCCTTC | 26 |
| | G | | | |
| PFKFβ3 | GGCAAGACCTACATCTCCAA | 27 | ATGGCTTCCTCATTGTCGG | 28 |
| | G | | | |
| GLUT1 | CATCAACCGCAACGAGGA | 29 | GGTCATGGGTCACGTCAG | 30 |
| PKM | ATGGCTGACACATTCCTGG | 31 | CATCTCCTTCAACGTCTCCAC | 32 |
| SIRT1 | CCCTCAAAGTAAGACCAGTA | 33 | CACAGTCTCCAAGAAGCTCTA | 34 |
| | GC | | C | |
| HIF1α | CCGCTGGAGACACAATCATA | 35 | ACTTCCTCAAGTTGCTGGTC | 36 |
| | TC | | | |
| EPAS1 | CCCATGTCTCCACCTTCAAG | 37 | GGCTTGCTCTTCATACTCCAG | 38 |
| GSK3β | GGTCTATCTTAATCTGGTGCT | 39 | TGGATATAGGCTAAACTTCGGA | 40 |
| | GG | | AC | |
| BMP2 | CTACATGCTAGACCTGTATCG | 41 | CCCACTCGTTTCTGGTAGTTC | 42 |
| | C | | | |
| PAX6 | GCCCTCACAAACACCTACAG | 43 | TCATAACTCCGCCCATTCAC | 44 |
| SNAI1 | GGAAGCCTAACTACAGCGAG | 45 | CAGAGTCCCAGATGAGCATTG | 46 |
| CD34 | AGAAAGGCTGGGCGAAGAC | 47 | TAGCACGTGGTCAGATGCAG | 48 |
| JAK1 | AACCTCTTTGCCCTGTATGAC | 49 | CTGCTCATTGTCGTTGGTTC | 59 |
| NOTCH1 | TGCCTGGACAAGATCAATGA | 51 | CAGGTGTAAGTGTTGGGTCC | 52 |
| | G | | | |
| STAT3 | TTCTGGGCACAAACACAAAA | 53 | TCAGTCACAATCAGGGAAGC | 54 |
| | G | | | |
| TWIST1 | CTCAGCTACGCCTTCTCG | 55 | ACTGTCCATTTTCTCCTTCTCT | 56 |
| | | | G | |
| YAP1 | ACAAGCCATGACTCAGGATG | 57 | TGTTTCACTGGAGCACTCTG | 58 |
| RUNX1 | CCAGGTTGCAAGATTTAATGA | 59 | TTTTGATGGCTCTGTGGTAGG | 60 |
| | CC | | | |
| GATA3 | GCGGGCTCTATCACAAAATG | 61 | TCCCCATTGGCATTCCTC | 62 |
| SOX7 | CACAACGCCGAGCTCAG | 63 | GGCCGGTACTTGTAGTTGG | 64 |
| TEK | CTGGGTTTATGGGAAGGACG | 65 | CAGGGAGACAGAACACATAAG | 66 |
| | | | AC | |
| VEGF-A | CGAGTACATCTTCAAGCCATC | 67 | TGGTGAGGTTTGATCCGC | 68 |
| | C | | | |
| Klf2 | CCTACACCAAGAGTTCGCAT | 69 | TGTGCTTTCGGTAGTGGC | 70 |
| | C | | | |
| HEY1 | TGGTACCCAGTGCTTTTGAG | 71 | CTCCGATAGTCCATAGCAAGG | 72 |
| MALL | CTGTTCCTCACCATCCCTTTC | 73 | CAAGGAGATGAGAAACGAGGT | 74 |
| | | | G | |
| ESM1 | GGTGTCAGCCTTCTAATGGG | 75 | TCAGGCATTTTCCCGTCC | 76 |
| WARS | TCAGCAACTCATTCCCACAG | 77 | GCAGGGCTGGTTTAGGATAG | 78 |
| JAG1 | GGACTATGAGGGCAAGAACT | 79 | AAATATACCGCACCCCTTCAG | 80 |
| | G | | | |

Immunofluorescence Staining

Cells were fixed with 4% paraformaldehyde for 15 min, permeabilised with 0.1% Triton X-100 in PBS for 5 min and blocked in 5% donkey serum in PBS for 30 min at room temperature. Cells were incubated with primary antibodies for 1 h at 37° C., the antibodies being: rabbit anti-VE-cadherin; rabbit anti-CD31 (human specific); rabbit anti-KDR; and mouse anti-SM22. The following incubation with the secondary antibodies was performed for 45 min at 37° C., using anti-rabbit Alexa488 and anti-mouse Alexa594. Cells were counterstained with 4′-6-diamino-2-phenylindole (DAPI), mounted on glass slides and examined with a fluorescence microscope (Axioplan 2 imaging; Zeiss) or SP5 confocal microscope (Leica, Germany).

Immunoblotting

Cells were harvested and washed with cold PBS, re-suspended in RIPA buffer (Sigma) and lysed by ultrasonication (twice, 6 seconds each) (Bradson Sonifier150) to obtain whole cell lysate. The protein concentration was determined using the Biorad Protein Assay Reagent. 50 μg of whole lysate was applied to SDS-PAGE and transferred to Hybond PVDF membrane (GE Health), followed by standard immunoblotting procedure. The bound primary antibodies were detected by the use of horseradish peroxidase (HRP)-conjugated secondary antibody and the ECL detection system (GE Health).

FACS Analysis

iPS-ECs were analysed with FACS to determine the percentage of CD144, KDR and other endothelial markers in the flow cytometer. Data analysis was performed using FlowJo software.

Ac-LDL Uptake Assay

To detect acetylated low-density lipoprotein (LDL) uptake, cells were incubated with Dil-ac-LDL (Molecular Probes) for 4 h and were examined and photographed under a fluorescence microscope (Axioplan 2 imaging; Zeiss).

In Vitro Tube Formation Assay

Cell suspensions containing 5×10⁴ iPS, iPS-ECs or HUVECs were placed on top of 50 μl/well Matrigel (BD Matrigel Basement Membrane Matrix Growth Factor Reduced) in 8-well chamber slides and incubated for 30-60 min at 37° C. to allow the gel to solidify.

Results and Discussion TERT1 is a Key Mediator of iPS Cells Reprogramming

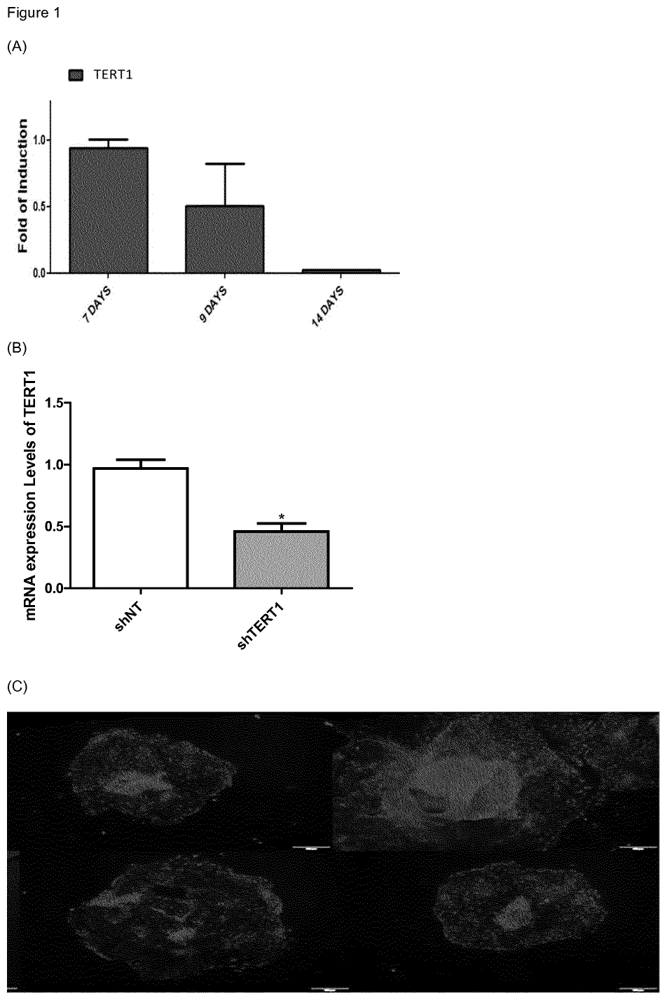

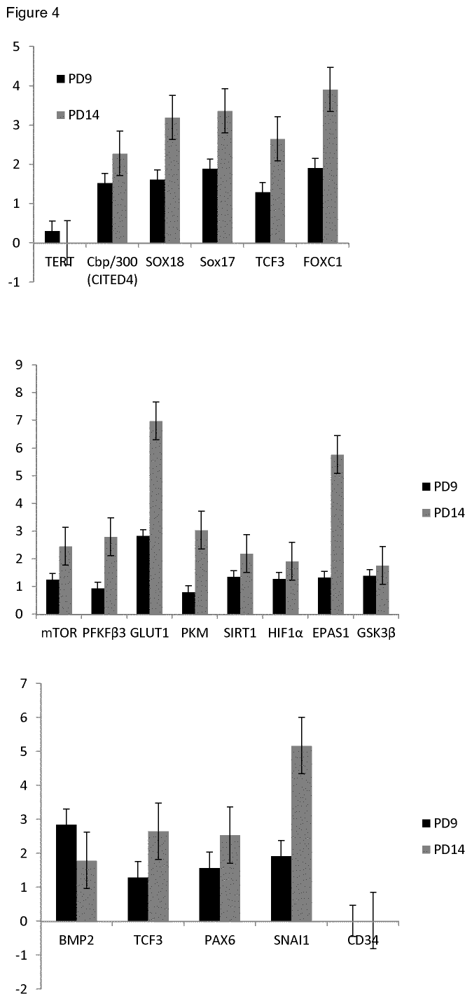

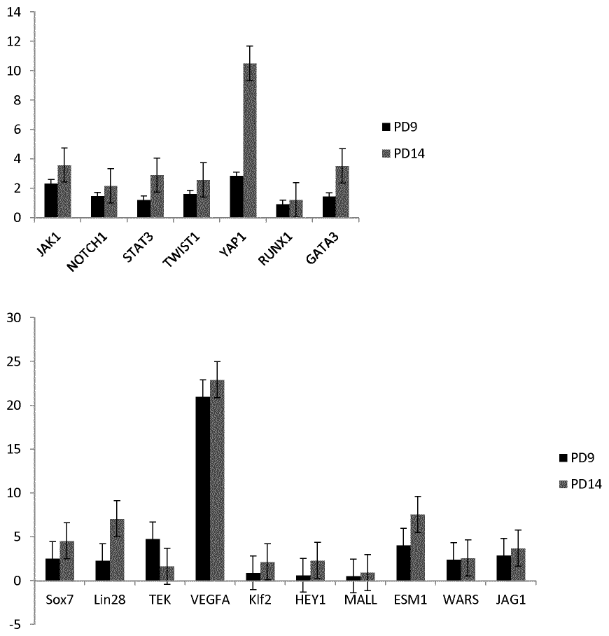

In an attempt to develop a robust protocol of generation of induced pluripotent stem cell from health volunteers and diabetic patients, mononuclear cells (MNCs) have been isolated from 1 ml of blood. In order to define the best time point of the mononuclear cell expansion which makes the cells responsive to cell reprogramming, a large screening assay profiling of monocular cells on days 9 and 14 was performed (FIG. 4). This large screening assays including reprogramming genes, epigenetic modulators, vascular progenitors, and early and late endothelial cell lineage genes. Remarkably, telomerase reverse transcriptase (TERT1), which is seems to have an extratelomeric 35 function in somatic cell reprogramming has been found to be a key mediator of iPS cells reprogramming. Telomerase reverse transcriptase (abbreviated to TERT, TERT1, or hTERT in humans) is a catalytic subunit of the enzyme telomerase, which, together with the telomerase RNA component (TERC), comprises the most important unit of the telomerase complex. As indicated in FIG. 1A, the expression levels of TERT1 were totally abolished after 14 days of cultured MNCs (FIG. 1A), at which time-point reprogramming efficiency is almost zero. Table 2 below demonstrates colony formation after 7 days expansion, but only a single colony was formed from MNCs expanded for 14 days.

TABLE 2
Comparison of iPSC colony formation after 7 and 14 days expansion.

| | MNCs - day 7 | MNCs - day 14 |
|---|---|---|
| Number of colonies | 8 | 1 |
| Cells seeded | 280 000 (in 3 wells) | 280 000 (in 3 wells) |

In order to confirm the role of TERT1 in reprogramming of cells, TERT1 was knocked down in 7-day MNCs by shRNA (FIG. 1B) and the reprogramming efficiency was reduced to 0% (Table 3) thus indicating that TERT1 expression is an important mediator of iPS cell generation. Control MNCs with normal TERT1 levels generated iPS cell colonies (Table 3), which cells express pluripotency stem cell markers such as CDy1 (FIG. 1C). Based on these findings, a new protocol of generating pluripotent stem cells has been developed. MNCs obtained from only few drops of blood may be expanded for only 7 days and subjected to iPS reprogramming. This protocol generates iPS cells colonies, for the first time, within one week in a reproducible and highly efficient manner. The establishment of such novel and short protocols are powerful tools for personalised and regenerative medicine.

TABLE 3
Generation iPS cell colonies in control and TERT1 knockdown cells.

| | Cells | | |
|---|---|---|---|
| Sample | reprogrammed | Number of colonies | Efficiency |
| CONTROL | 1 000 000 | 6 | 0.002% |
| TERT1 knockdown | 1 000 000 | 0 | 0.000% |

Generation and Characterisation of iPS Cells Obtained from Few Drops of Blood Based on a Novel Approach

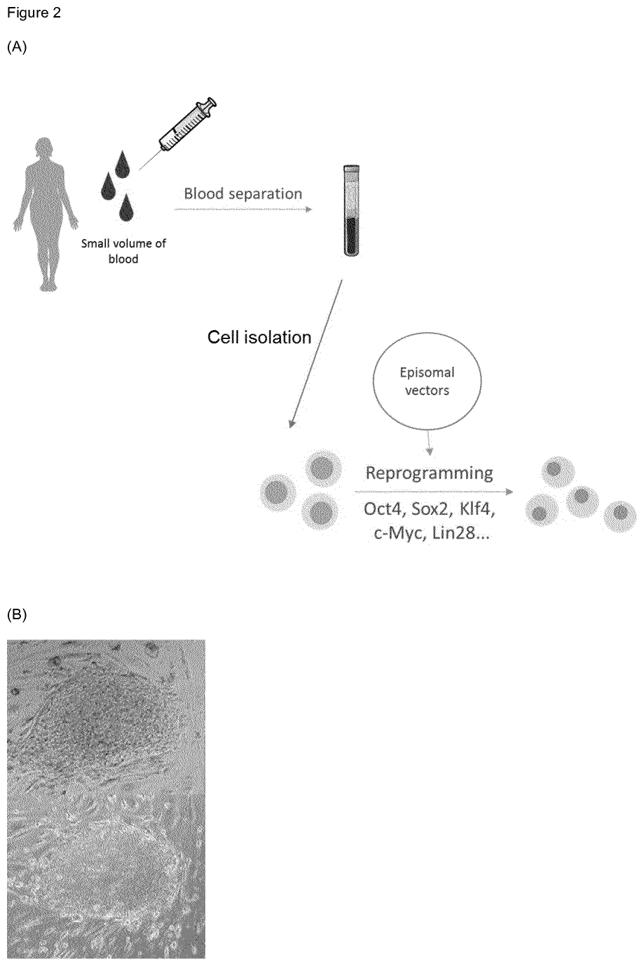

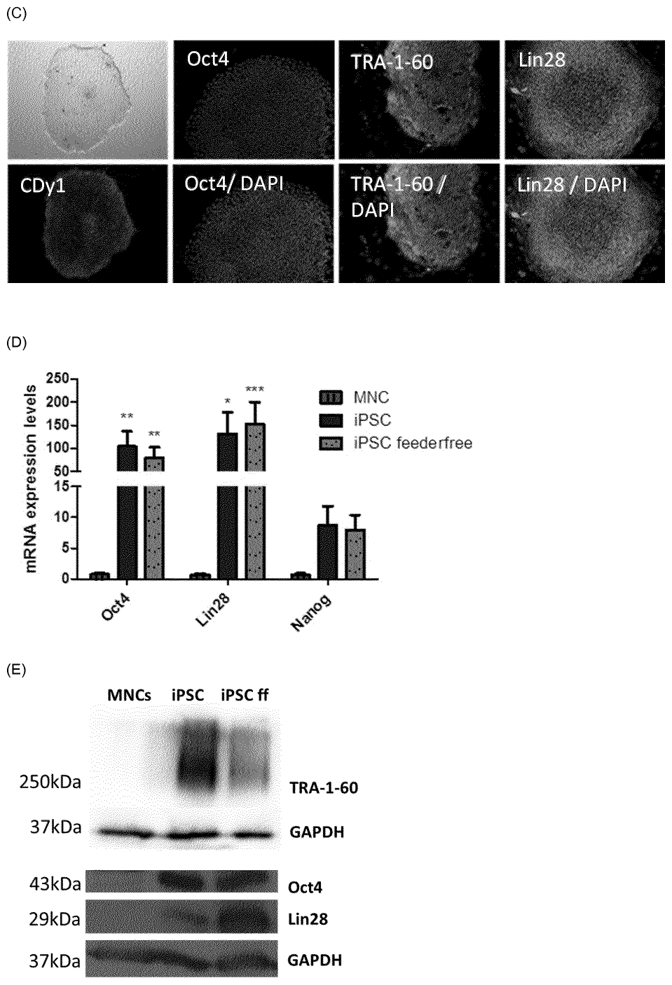

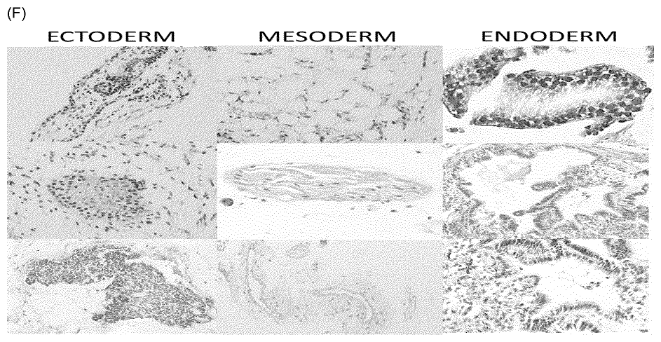

This powerful, fast and highly efficient reprogramming method from few drops of blood has been generated and the derived iPS cells have been fully characterised. In FIG. 2A, a diagram is shown explaining the iPSC generation from few drops of blood samples from health volunteers and diabetic patients. iPS cells formed round colonies with defined limits revealing a typical morphology of pluripotent stem cells (FIG. 2B). Immunofluorescence staining has been performed showing that iPS cells express pluripotent markers such as CDy1 (in vitro live staining), Oct4, TRA-1-60 and Lin28—see FIG. 2C. Moreover, iPS cells express Oct4, Lin28 and Nanog at an mRNA level (FIG. 2D). Importantly, iPS cells express TRA-1-60, Oct4 and Lin28 at a protein level (FIG. 2E). Finally, the pluripotency of the iPS cells derived based on our novel approach was tested in vivo by performing teratoma formation in SCID Mice. Indeed, the iPS cells formed teratomas within 6-8 weeks in vivo (FIG. 2F). These results highlight that iPS cells are generated based on a novel, fast, and highly efficient method from few drops of blood from health volunteers and diabetic patients.

Experiments were conducted to identify a suitable feeder-free and xeno-free substrate on which to grow and reprogramme somatic cells (MNCs) to iPS cells. Various concentrations of gelatin and laminin were mixed with a suitable buffer (phosphate buffered saline) and allowed to set. As noted in Table 4, we discovered that only certain substrates comprising a combination of gelatin and laminin, in the indicated quantities, produced a suitable gel consistency and produced colonies of iPS cells.

TABLE 4
iPS cell colony formation using different formulations of substrates.

| | Concentration Range | | | | | |
|---|---|---|---|---|---|---|
| Gelatin | 0.02% | 0.04%-0.1 | 0.5-0.8% | 1-5% | 1-5% | >10% |
| | Gel is not | Gel is not | Gel is | Gel is | Gel is | Gel is |
| | formed | fully formed | formed | formed | formed | crystalized |
| Laminin | 1-10 μg/ml | 50-100 μg/ml | 50-100 μg/ml | 50-100 μg/ml | 200 μg/ml | 50-100 μg/ml |
| Colonies | No colonies | Few colonies | Some | Many | Very few | No colonies |
| | | | colonies | colonies | colonies | |

Experiments were also conducted using feeder-free media with modifications in the coating substrate, seeding density, transfection method and/or use of high passage MNCs. No colonies were obtained using high passage cells (i.e. somatic cells (MNCs) expanded for days before reprogramming). Also, no colonies were obtained using other transfection methods such as Lipofectamine™ and Fugene® 6 (data not shown). A seeding density of about 1-2×10⁶ cells per well of 6-well plate produced best results for reprogramming efficiency. As noted from Table 5, standard coating substrates produced no, or very few, iPS cell colonies whereas our novel substrates produced colonies after only about 5-7 days.

TABLE 5
iPS cell colony formation using different plating substrates.

| Substrate | Colonies |
|---|---|
| (ES) Matrigel | No |
| Gelatin with Matrigel | No |
| Cell Matrix | No |
| Matrigel with Cell Matrix | No |
| Matrigel* | Few (and delayed appearance) |
| Novel substrate** | Yes |

*Standard protocol based on viruses as disclosed in Kishino et al. [7].
**Produced as described above under “Materials and Methods”.

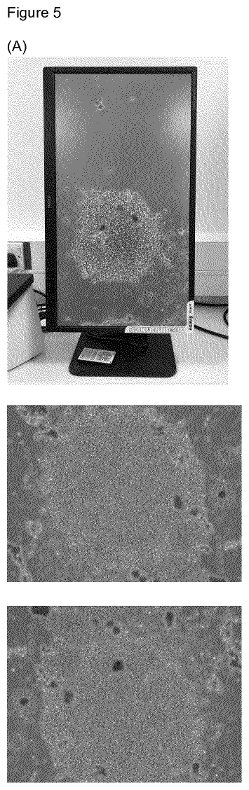

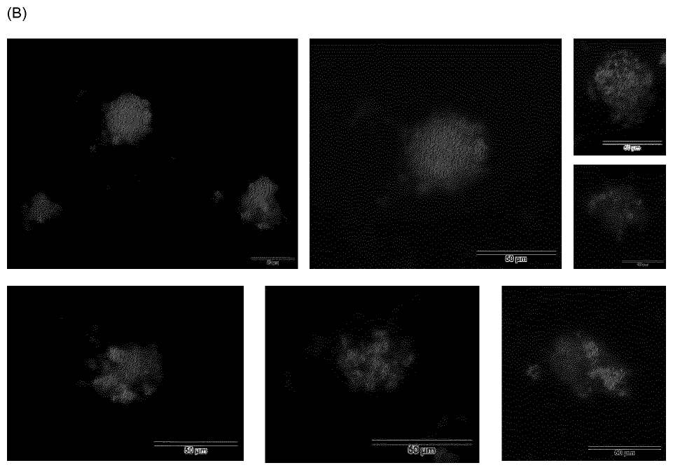

Thus, at around day 5-7, typical iPS cell colonies with well-defined round limits were observed on the novel substrate (see FIG. 5A), through the assessment of extensively characterised pluripotency-associated markers. The iPS cell colonies stained positive for the pluripotent marker CDy1 (FIG. 5B).

Differentiation of the iPS Cells Towards Functional Endothelial Cells

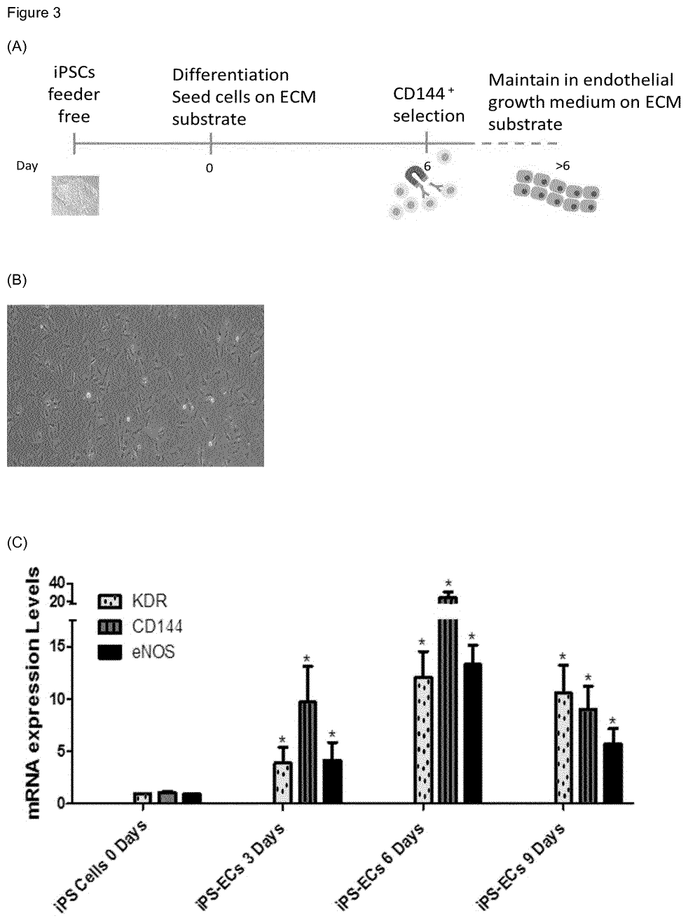

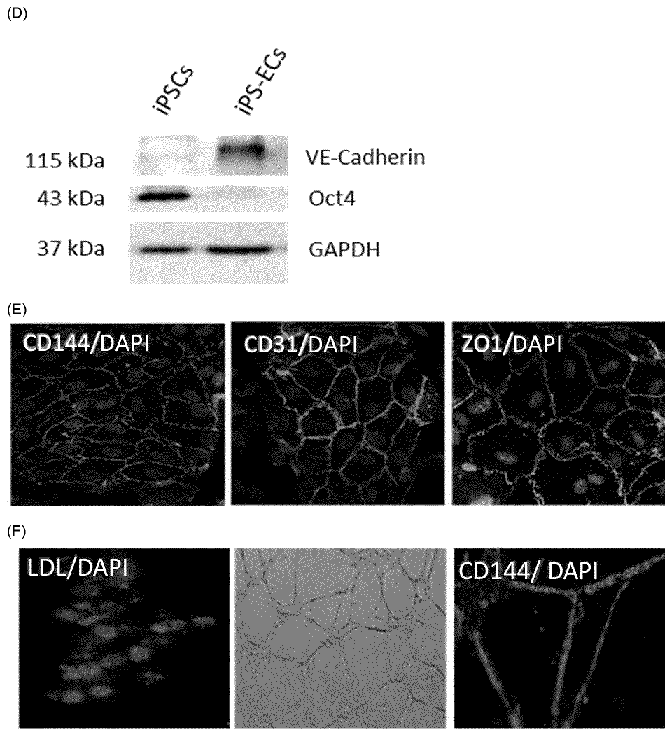

The next step was to differentiate the iPS cells towards endothelial cell (EC) lineages. In FIG. 3A, a schematic diagram of iPS cell differentiation towards ECs is shown. Briefly, iPS cells were cultured under feeder-free conditions and seeded on mouse collagen IV-coated plates in EGM-2 media (Lonza) 10% FBS. The medium was supplemented with 25 ng/ml BMP4, 12 ng/ml Activin A, 8 μM CHIR99021, and 20 ng/ml FGF2. After 48 hours (day 2), the medium was replaced with EGM-2 plus 10% FBS supplemented with 50 ng/ml VEGF, 10 ng/ml FGF₂ and 10 μM LY364947 (Sigma) and refreshed every other day. In day 6 of differentiation, MACS-mediated selection for CD144-expressing cells was performed and the selected cells were seeded on mouse collagen IV-coated plates. The media used was EGM-2 10% FBS supplemented with 50 ng/ml VEGF and 10 μM LY364947. The derived iPS-ECs reveal a typical morphology of ECs (FIG. 3B). Real time data are also shown that iPS-ECs express EC markers such as KDR, CD144, eNOS at the mRNA level (FIG. 3C). Importantly, upon EC differentiation, iPS-ECs express VE-cadherin at a protein level and stop expressing the pluripotent marker OCT4 (FIG. 3D). Remarkably, iPS-ECs form tight junctions in vitro and express VE-cadherin, PECAM-1 and ZO-1 (FIG. 3E). Functionally, iPS-ECs take up LDL and they form vascular structures in in vitro Tube Formation Assays (FIG. 3F). These results clearly demonstrate that functional ECs are derived and are powerful tools for drug screening and cell based therapies.

In a further development, the human induced pluripotent stem (iPS) cells cultured under feeder-free conditions were detached using dissociation solution (Reprocell) and seeded on mouse collagen IV (Cultrex Mouse Collagen IV (3410-010-01, R&D) in EGM-2 media (Lonza) 10% FBS. The medium was supplemented with 25 ng/ml BMP4, 12 ng/ml Activin A, 8 μM CHIR99021 and 20 ng/ml FGF2 (MACS). After 48 hours (day 2 of differentiation), the medium was replaced with EGM-2 10% FBS supplemented with 200 ng/ml VEGF (Life Technologies), 10 ng/ml FGF₂ and 10 μM LY364947 (Sigma) and was refreshed every other day. On day 6 of differentiation, MACS-mediated magnetic selection for CD144-expressing cells was performed using MicroBeads Kit (Miltenyi Biotec), as we have previously shown (Cochrane et al., 2017). The positively selected cells were seeded on mouse collagen IV-coated plates in EGM-2 10% FBS media supplemented with 50 ng/ml VEGF and 10 μM LY364947.

Discussion

In this study, we developed, for the first time, a reprogramming method to generate iPS cells based on a novel, fast and highly efficient strategy. Our laboratory has extensive experience in reprogramming somatic cell populations (fibroblasts or mononuclear cells) to iPS cells [3], as well as, partial-iPS cells (PiPS) [4]. The process uses somatic cells from diabetic patients and healthy controls, making use of a DNA-free integration technique. In this study, we have gone further by proposing a novel and short approach to iPS cells reprogramming. Stem cell based therapies represent an emerging field within medical research, with the potential to revolutionize health care, offering the ability to apply personalised medicine, without the associated risks of tissue rejection or use of immunosuppressive drugs. By definition, stem cells harbour a high self-renewal potential, and innate ability to differentiate into a multitude of different cell types, depending upon their relative surrounding chemical milieu, or “niche”. The applications of stem cells are as far reaching as treating cardiovascular disease (CVD), various malignancies, and Alzheimer's disease. In general, stem cells can be classified according to their potency, that is, their relative ability to differentiate into the various cell types of the body. Those described as pluripotent are capable of forming all cell lineages of the body, while those of a multipotent state are yet further terminally differentiated, and are, as consequence, more restricted with regards to the repertoire of cell types that they can adopt. It would therefore follow that those cells of greatest potency are of most use to cell-based treatment strategies. Embryonic stem cells represent the only natural pluripotent human stem cells, and are generally isolated following somatic cell nuclear transfer and extraction from the inner cell mass of the resultant blastocyst. Nonetheless, there remains a number of barriers in the application of the former cells, including concerns regarding tumorigenesis, the relative supply of human embryos and ethical apprehensions of the general public. However, it has been reasoned by scientists that the very same factors responsible for the maintenance of pluripotency in embryonic stem cells could potentially prompt such potency in somatic cells. Remarkably, it was found that the combination of only four select factors was sufficient to generate induced pluripotent stem (iPS) cells from mouse fibroblast cultures. These factors included the gene regulatory proteins, Oct3/4, Sox2, Klf4, and c-Myc. It was subsequently found that the generation of human iPS cells from adult human dermal fibroblast was possible, again through ectopic expression of the same four factors. Subsequent analysis revealed that human iPS cells shared a number of distinctive features with human embryonic cells, including proliferative potential, epigenetic status of pluripotent cell-specific genes, and of great importance for medical application, the ability to generate all three germ layers. Such findings were instrumental in providing conclusive evidence that human iPS cells can indeed be generated from somatic cell lines. Other work re-affirmed the capacity of human somatic cells to adopt a pluripotent state upon expression of these seemingly quintessential factors. Incredibly, in 2009, it was reported that over-expression of a single Yamanaka transcription factor, Oct4, was sufficient for the reprogramming of adult mouse neural stem cells into iPS cells. A comprehensive description of the exact global targets and signal networks regulated by the Yamanaka factors was defined by the combined work of two later studies in which the precise cis-acting targets of nine transcription factors was identified, including the aforementioned factors, and further target promoters were identified and extended this work to analysis of the signalling cascades controlled by the Yamanaka factors in mouse embryonic cells. The sheer complexity of these pathways can be appreciated when considering the array of signalling cascades involved. With regard to the field of regenerative medicine, these cells harbour significant potential, offering the prospect to treat some of the most debilitating diseases affecting modern society; it may very well be the case that their application is limited only by our creativity. Beyond their use in a clinical environment, iPS cells may represent an invaluable tool for disease modelling and novel drug screening trials.

Re-Programming of Somatic Cells

Within the past decade, several methods have been employed to successfully produce iPS cells from various human somatic cell lines. Yet more complex vectors were devised and produced in an effort to improve retroviral gene transfer; namely vectors based upon the lentiviruses, and adopted in further studies.

Despite the apparent success of the aforementioned protocols, retroviral vectors are incredibly inefficient, creating substantial genetic heterogeneity in the infected somatic cells, with as little as 0.001 to 0.1% of the cells acquiring a subsequent pluripotent state. In light of these inefficiencies, a drug-inducible lentivirus vector system was developed, and reported increased efficacies, in comparison to early methods, of over two orders of magnitude. Nevertheless, the fact that many of the reprogramming factors are oncogenes, and the use of retroviral vectors increases the risk of insertional mutagenesis, confers an appreciable risk of oncogenic transformation. As a result, iPS cell generation with retroviral vectors is only appropriate when applied to in vitro studies, such as modelling the pathogenesis of disease. It could be inferred that iPS cell production with fewer factors could potentially evade tumorigenesis, and indeed select studies have demonstrated the successful exclusion of the c-myc oncogene. This, however, is not without consequence, as a subsequent decline in re-programming efficiency begets an increase in the accumulation of deleterious mutations.

Non-integrating lentiviral vectors were thus developed, generating more fitting therapeutic iPS cells, with a reduction in insertional mutagenesis and a concurrent decline in the development of malignancies amongst cell lines.

Therefore, more advanced strategies are urgently needed to generate iPS cells in a shorter timeframe and a more efficient manner. In the present study, we have found that 7-days MNCs express high levels of the epigenetic gene TERT1, which makes the cells respond to cell reprogramming and “be transformed” quickly and efficiently towards iPS cells in very short time. For the first time, a robust method of generating iPS cells using cells isolated from a tiny amount of blood has been demonstrated. However, while iPS cells can be derived from blood cells, there is a major problem relating to low efficiency of reprogramming and safety which relates to requirement for large blood volumes and also, in some instances, drug-induced mobilisation of blood cells using granulocyte-macrophage colony stimulating factor (GM-CSF). Currently, there are a limited number of blood-based kits that can generate iPS cells in a fast and safe way. In this study, we have developed a novel approach of iPS cells generation, which is less invasive. The use of blood mononuclear cells (MNCs) for patient/donor-specific cell reprogramming is less invasive for the subject than the use of fibroblasts and requires only as little as 1 ml of blood, and importantly, without blood mobilisation. It does not require previous growth factor treatment of the donor and the peripheral blood is extracted by venipuncture. There is applicability in both healthy donor and diabetic patient samples. This is especially important for diabetic patients who have healing difficulties due to their condition. It is a rapid approach: we are able to reprogram cells from very small volume of blood cells (1 ml), which are expanded in only 7 days and generate fully reprogrammed iPS cells colonies only 9 days after reprogramming. It is a faster and more efficient expansion: MNCs can be expanded with an up to 2.5-fold increase in 7 days. It is highly efficient: the reprogramming efficiency of the MNCs is up to 0.02%, which is high when compared to the efficiency obtained by other protocols that use integration free and virus-free gene delivery methods. The establishment of fully reprogrammed iPS cells colonies is reported throughout the present protocol: this is very important since other protocols can lead to generation of partially reprogrammed cells that fail in the characterisation or differentiation steps. The iPS cells obtained with this method have been characterised as fully reprogrammed pluripotent stem cells and have been successfully differentiated towards ECs. The present method is virus-free: no need for use of a virus to deliver the reprogramming genes into the cells, and non-integrating episomal vectors can be used instead. This reduces any mutations in the reprogrammed cells and makes them safer for regenerative medicine uses. The present method is feeder-free: no need for use of feeder cells such as mouse embryonic fibroblasts (MEFs), which are commonly used when growing human iPS cells. Thus, there is no external contamination risk, which is a great advantage when studying human diseases or making it capable of translation into patient treatment and applicable for future clinical use. It is cost effective: the replication and use of episomal vector is highly cost-effective when compared to other non-integrating gene delivery methods for cell reprogramming such as Sendai virus or episomal vector transfection kits. Finally, it is a flexible method: the production and use of the vectors do not need a specific kit.

The Method can be Used Widely to Generate Patient Specific iPS Cells for Drug Screening and Cell Based Therapies

Importantly, in this study, iPS cells from diabetic patients have been generated and differentiated toward ECs. It is projected that by 2025, there will be 380 million people with diabetes, making diabetes the 7^{th} leading cause of death by 2030. Diabetes is a major global cause of premature mortality. Diabetes is a major cause of heart attacks, stroke, lower limb amputation, kidney failure and blindness, all are devastated conditions which severely affect the quality of life and cause death. Approximately one half of patients with type 2 diabetes die prematurely of a cardiovascular cause and approximately 10% die of renal failure. The pathogenic basis for macro- and micro-vascular complications arising from both Type 1 and Type 2 diabetes is complex and multifactorial, but a progressive EC dysfunction is critical. As a consequence of the hyperglycaemia, hypertension and dyslipidaemia which characterise the diabetic milieu, a vicious circle of events can occur in the vascular endothelium involving oxidative stress, low-grade inflammation and platelet hyperactivity. How hyperglycaemia in particular influences endothelial dysfunction remains incompletely understood. Therefore, there is a very urgent necessity for studies to be conducted based on pioneering ideas and novel tools such as the potential of deriving ECs from diabetic patients through the remarkable technology of the iPS cells. iPS cells hold an enormous potential with regards to targeting therapeutic strategies to the downstream causal factors of diabetes; that is pronounced EC dysfunction. Indeed, the generation of functional vascular tissue to replace that which has become lost or damaged in the process of diabetes and prevent diabetic complications, especially when considering the fact that the spontaneous regeneration of ECs is incredibly slow, may represent a possible breakthrough in what has been an exceedingly challenging disease to treat. In addition to this re-endothelization of blood vessels, cell-based therapies could also be directed towards vasculogenesis; supporting angiogenesis within ischemic tissues following acute myocardial infarction, which is a major complication of diabetes. Therefore, the derived ECs from a diabetic patient are unique tools which represent the patient-specific cells in a petii-dish which can be used for the first time to study the causes of the disease; to screen an unlimited number of potential drugs; to develop new therapies; and to generate functional cell to be used for cell based therapies. Diabetes and diabetic complication are only one example that our novel method could have an enormous impact. The list is endless and countless numbers of patients are waiting for novel treatments based on this promising and powerful strategy.

The invention is not limited to the embodiments described herein but can be amended or modified without departing from the scope of the present invention.

### REFERENCES

[1] Worringer K A, Rand T A, Hayashi Y, Sami S, Takahashi K, Tanabe K et al. “The let-7/LIN-41 pathway regulates reprogramming to human induced pluripotent stem cells by controlling expression of prodifferentiation genes.” Cell Stem Cell 2014; 14(1): 40-52.
[2] Takahashi K and Yamanaka S. “Induction of pluripotent stem cells from mouse embryonic and adult fibroblast cultures by defined factors.” Cell 2006; 126(4): 663-676.
[3] Cochrane, A., et al. “Quaking Is a Key Regulator of Endothelial Cell Differentiation, Neovascularization, and Angiogenesis.” Stem Cells (2017).
[4] Margariti, A., et al. “Direct reprogramming of fibroblasts into endothelial cells capable of angiogenesis and reendothelialization in tissue-engineered vessels.” Proc Natl Acad Sci USA 109, 13793-13798 (2012).
[5] Chou, B.-K., Mali, P., Huang, X., Ye, Z., Dowey, S. N., Resar, L. M. S., Zou, C., Zhang, Y. A., Tong, J., and Cheng, L. (2011). Efficient human iPS cell derivation by a non-integrating plasmid from blood cells with unique epigenetic and gene expression signatures. Cell Research 21, 518.
[6] Dowey, S. N., Huang, X., Chou, B.-K., Ye, Z., and Cheng, L. (2012). Generation of integration-free human induced pluripotent stem cells from postnatal blood mononuclear cells by plasmid vector expression. Nature Protocols 7, 2013.
[7] Kishino et al., “Generation of Induced Pluripotent Stem Cells from Human Peripheral T Cells Using Sendai Virus in Feeder-free Conditions”, J Vis Exp. 2015; (105): 53225

1. A composition for promoting the reprogramming of somatic cells to induced pluripotent stem cells, the composition comprising gelatin at a concentration of about 0.01 w/v % to about 10 w/v % and laminin at a concentration of about 1 μg/mL to about 1000 μg/mL.

2. The composition of claim 1, wherein the composition comprises gelatin at a concentration of about 0.04 w/v % to about 10 w/v %, optionally about 0.04 w/v % to about 5 w/v %, and laminin at a concentration of about 50 μg/mL to about 200 μg/mL.

3. The composition of claim 1 or 2, wherein the composition comprises gelatin at a concentration of about 0.04 w/v % to about 10 w/v %, optionally about 0.04 w/v % to about 5 w/v %, and laminin at a concentration of about 50 μg/mL to about 100 μg/mL.

4. The composition of claim 1 or 2, wherein the composition comprises gelatin at a concentration of about 0.5 w/v % to about 10 w/v %, optionally about 0.5 w/v % to about 5 w/v %, and laminin at a concentration of about 50 μg/mL to about 200 μg/mL.

5. The composition of any one of the preceding claims, wherein the composition comprises gelatin at a concentration of about 0.5 w/v % to about 10 w/v %, optionally about 0.5 w/v % to about 5 w/v %, and laminin at a concentration of about 50 μg/mL to about 100 μg/mL.

6. The composition of claim 1 or 2, wherein the composition comprises gelatin at a concentration of about 1 w/v % to about 10 w/v %, optionally about 1 w/v % to about 5 w/v %, and laminin at a concentration of about 50 μg/mL to about 200 μg/mL.

7. The composition of any one of the preceding claims, wherein the composition comprises gelatin at a concentration of about 1 w/v % to about 10 w/v %, optionally about 1 w/V % to about 5 w/v %, and laminin at a concentration of about 50 μg/mL to about 100 μg/mL.

8. The composition of any one of the preceding claims, wherein the laminin is recombinant human laminin.

9. The composition of any one of the preceding claims, wherein the gelatin is recombinant human gelatin.

10. The composition of any one of the preceding claims, wherein the composition is an aqueous composition, optionally wherein the aqueous composition comprises, or consists of, a liquid or a gel.

11. The composition of any one of the preceding claims, wherein the composition is provided as a dry, or substantially dry, composition which may be formed into an aqueous composition by the addition of a solvent.

12. The composition of claim 11, wherein the solvent is selected from one of more of saline, optionally phosphate buffered saline; cell culture medium; and water, optionally sterile water.

13. The composition of any one of the preceding claims, wherein the composition further comprises one or more additional extracellular matrix components.

14. The composition of claim 13, wherein the one or more additional extracellular matrix components are selected from collagen, elastin, fibronectin, nidogen, and heparan sulfate proteoglycan.

15. The composition of any one of the preceding claims, wherein the composition further comprises one or more growth factors.

16. The composition of claim 15, wherein the one or more growth factors are selected from one or more of transforming growth factor beta (TGF-beta) epidermal growth factor (EGF), insulin-like growth factor (IGF), and fibroblast growth factor (FGF).

17. The composition of any one of the preceding claims, wherein the composition further comprises a Rho-associated protein kinase (ROCK) inhibitor.

18. The composition of claim 17, wherein the ROCK inhibitor is selected from one or more of Y-27632 dihydrochloride (trans-4-[(1R)-1-Aminoethyl]-N-4-pyridinylcyclohexanecarboxamide dihydrochloride), GSK429286A (N-(6-fluoro-1H-indazol-5-yl)-6-methyl-2-oxo-4-[4-(trifluoromethyl)phenyl]-3,4-dihydro-1H-pyridine-5-carboxamide), Y-30141 (4-(1-aminoethyl)-N-(1H-pyrrolo(2,3-b)pyridin-4-yl)cyclohexanecarboxamide dihydrochloride), RKI-1447 (N-[(3-Hydroxyphenyl)methyl]-N′-[4-(4-pyridinyl)-2-thiazolyl]urea dihydrochloride), Fasudil, and Ripasudil (trade name Glanatec).

19. The composition of claim 17 or 18, wherein the ROCK inhibitor is present in the composition at a concentration of about 1 μM to 1 mM, optionally about 1 μM to 100 mM, optionally about 1 μM to 1 mM, optionally about 1 μM to 100 μM, optionally about 1 μM to 50 μM, optionally about 5 μM to 50 μM, optionally about 10 μM to 50 μM, further optionally about 10 μM.

20. The composition of any one of the preceding claims, wherein the composition further comprises genetic elements, optionally episomal genetic elements, which comprise or consist of induced pluripotent stem cells reprogramming factors selected from one or more of Oct4, Sox2, Klf4, c-Myc, Lin28, Nanog and SV40 large T.

21. The composition of claim 20, wherein the genetic elements further comprise TERT1.

22. The composition of claim 20 or 21, wherein the genetic elements comprise nucleic acid sequences coding, optionally DNA nucleotide sequences, comprised in a plasmid or other vector suitable for transfection into somatic cells.

23. The composition of any one of the preceding claims, wherein the composition further a carrier which is suitable to deliver genetic elements inside somatic cells.

24. The composition of claim 23, wherein the carrier comprises nanoparticles for nanoparticle-mediated delivery of genetic elements to the somatic cells.

25. The composition of claim 23 or 24, wherein the carrier comprises lipid-based nanoparticles, optionally wherein the lipid-based nanoparticles nanoparticles comprise liposomes, optionally cationic liposomes.

26. Use of the composition of any one of claims 1 to 25 in a method for reprogramming of somatic cells to induced pluripotent stem cells.

27. A cell culture vessel comprising the composition of any one of claims 1 to 25.

28. A kit comprising the composition of any one of claims 1 to 25.

29. The kit of claim 28, further comprises a cell culture vessel.

30. The kit of claim 29, wherein the cell culture vessel comprises the composition.

31. A method of reprogramming somatic cells to induced pluripotent stem cells, the method comprising
(i) contacting the somatic cells with the composition of any one of claims 1 to 25;
(ii) introducing genetic elements, optionally episomal genetic elements, that express induced pluripotent stem cells reprogramming factors into the somatic cells; and
(iii) culturing said expanded somatic cells comprising the genetic elements, thereby producing induced pluripotent stem cells.

32. The method of claim 31, wherein, in step (i), the somatic cells are suspended in cell suspension medium when contacted with the composition.

33. The method of claim 32, wherein contacting the somatic cells suspended in the suspension medium with the composition causes the suspension medium to dissolve the composition.

34. The method of any one of claims 31 to 33, wherein the somatic cells comprise peripheral blood mononuclear cells, optionally wherein said peripheral blood mononuclear cells comprise monocytes.

35. The method of any one of claims 31 to 35, wherein the genetic elements that express induced pluripotent stem cells reprogramming factors are introduced into the somatic cells via the nanoparticles comprised in the composition.

36. The method of any one of claims 31 to 36, wherein the induced pluripotent stem cells produced from the somatic cells comprising the genetic elements are differentiated to endothelial cells.

37. A method of preparing somatic cells for producing induced pluripotent stem cells, the method comprising:
(i) isolating somatic cells from a sample, and
(ii) expanding the somatic cells for a predetermined period of time, wherein the expanded somatic cells express TERT1.

38. The method of claim 37, wherein the somatic cells are expanded for less than about 14 days, optionally less than about 13 days, optionally less than about 12 days, optionally less than about 11 days, optionally less than about 10 days, optionally less than about 9 days, optionally less than about 8 days, optionally less than about 7 days, further optionally about 7 days.

39. The method of claim 37 or 38, wherein the somatic cells are expanded for at least about 1 day, optionally at least about 2 days, optionally at least about 3 days, optionally at least about 4 days, optionally at least about 5 days, optionally at least about 6 days, further optionally at least about 7 days.

40. The method of any one of claims 37 to 39, wherein TERT1 expression is at least about 10%, optionally at least about 20%, optionally at least about 30%, optionally at least about 40%, optionally at least about 50%, optionally at least about 60%, optionally at least about 70%, optionally at least about 80%, optionally at least about 90%, optionally about 100%, of the expression of TERT1 in the somatic cells prior to expansion.

41. The method of any one of claims 37 to 40, wherein the sample is a biological sample obtained from a subject, optionally wherein the sample is a blood sample obtained from a subject.

42. The method of any one of claims 37 to 41, wherein the somatic cells are peripheral blood mononuclear cells, optionally wherein said peripheral blood mononuclear cells are monocytes.

43. The method of claim 41 or 42, wherein the volume of said blood sample is less than about 10 ml, optionally less than about 5 ml, optionally less than about 2.5 ml, optionally less than about 1 ml, further optionally about 1 ml.

44. The method of any one of claims 41 to 43, wherein the subject is a human subject, optionally wherein said subject suffers from diabetes.

45. The method of any one of claims 42 to 44, wherein the peripheral blood mononuclear cells have not been mobilized, optimally wherein the peripheral blood mononuclear cells have not been mobilized with extrinsically applied granulocyte colony stimulating factor (G-CSF) or granulocyte macrophage colony-stimulating factor (GM-CSF).

46. The method of any one of claims 37 to 45, wherein the somatic cells are expanded in an expansion medium, optionally wherein the expansion medium comprises serum free medium (SFM) supplemented with one or more of erythropoietin (EPO), IL-3, stem cell factor (SCF), insulin-like growth factor-1 (IGF-1), dexamethasone, and holo-transferrin.

47. The method of claim 46, wherein the somatic cells are expanded in the expansion medium for a first expansion period of about 1 to 7 days, optionally about 2 to 6 days, optionally about 2 to 5 days, optionally about 2 to 4 days, optionally 2 to 3 days, further optionally about 3 days.

48. The method of claim 47, wherein the somatic cells are expanded in the expansion medium for a second expansion period of about 1 to 7 days, optionally about 1 to 6 days, optionally about 1 to 5 days, optionally about 1 to 4 days, optionally 1 to 3 days, optionally 2 to 3 days, further optionally about 3 days.

49. The method of any one of claims 37 to 48, further comprising:
(iii) cryopreserving the expanded somatic cells.

50. The method of any one of claims 37 to 49, wherein TERT1 expression is measured in the expanded and/or unexpanded somatic cells, optionally wherein the expanded somatic cells are determined to be suitable for producing the induced pluripotent stem cells if the expanded somatic cells express TERT1.

51. A method for producing induced pluripotent stem cells, the method comprising:
(a) introducing genetic elements, optionally episomal genetic elements, that express induced pluripotent stem cells reprogramming factors into expanded somatic cells produced according to the method of any one of claims 37 to 50, and
(b) culturing said expanded somatic cells comprising the genetic elements, thereby producing induced pluripotent stem cells.

52. The method of claim 51, wherein the genetic elements are introduced into the expanded somatic cells via a non-viral transfection method, optionally via electroporation.

53. The method of claim 51 or 52 wherein the induced pluripotent stem cells reprogramming factors are selected from one or more of Oct4, Sox2, Klf4, c-Myc, Lin28, Nanog and SV40 large T.

54. The method of any one of claims 51 to 53, wherein, in step (b), the expanded somatic cells comprising the genetic elements are cultured in expansion medium, optionally for about 2 days.

55. The method of claim 54, wherein, following culturing in expansion medium, the expanded somatic cells comprising the genetic elements are then seeded onto inactivated mouse embryonic fibroblasts (MEFs), optionally for about 1 day.

56. The method of claim 55, wherein, following culturing on the inactivated mouse embryonic fibroblasts (MEFs), the expanded somatic cells comprising the genetic elements are removed from the MEFs and cultured in reprogramming medium comprising sodium borate, optionally for about 1 day.

57. The method of claim 56, wherein, the reprogramming medium comprising sodium borate is replaced with fresh reprogramming medium comprising sodium borate every day, optionally the reprogramming medium comprising sodium borate is replaced with fresh reprogramming medium comprising sodium borate every day for about 4-8 days, optionally about 5-7 days, optionally about 6 days.

58. The method of claim 57, wherein, the reprogramming medium comprising sodium borate is replaced with conditioned medium comprising sodium borate and basic fibroblast growth factor every day until one or more cell colonies comprising induced pluripotent stem cells are formed.

59. An expanded somatic cell produced according to the method of any one of claims 37 to 50.

60. An expanded somatic cell expressing TERT1.

61. The expanded somatic cell according to claim 60, wherein TERT1 expression in said expanded somatic cell is at least about 20%, optionally at least about 30%, optionally at least about 40%, optionally at least about 50%, optionally at least about 60%, optionally at least about 70%, optionally at least about 80%, optionally at least about 90%, optionally about 100%, of the expression of TERT1 in the unexpanded somatic cells.

62. The expanded somatic cell according to claim 61, wherein the TERT1 expression wherein the TERT1 expression is measured by real time polymerase chain reaction, reverse transcriptase quantitative polymerase chain reaction, western blotting and/or immunofluorescence microscopy.

63. The expanded somatic cell according to any one of claims 60 to 62, wherein the cell is produced according to the method of any one of claims 37 to 50.

64. An induced pluripotent stem cell produced according to the method of any one of claims 51 to 58.

65. An induced pluripotent stem cell produced from the expanded somatic cell of any one of claims 59 to 63.

66. The induced pluripotent stem cell of claim 65, wherein the induced pluripotent stem cell is produced according to the method of any one of claims 51 to 58.

67. An induced pluripotent stem cell according to any one of claims 64 to 66 for use in therapy.
